(19) United States
(12) Patent Application Publication
(10) Pub. No.: US 2004/0224963 A1
(43) Pub. Date: Nov. 11, 2004

(54) Pharmaceutical composition for the prevention and treatment of nicotine addiction in a mammal

(75) Inventors: Jotham W. Coe, Niantic, CT (US); Philip A. Iredale, Clinton, CT (US); Steven B. Sands, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/793,112

(22) Filed: Mar. 4, 2004

Related U.S. Application Data
(60) Provisional application No. 60/469,429, filed on May 9, 2003.

(51) Int. Cl. A61K 31/522; A61K 31/445

(52) U.S. Cl. 514/263.3; 514/317

(57) ABSTRACT

Pharmaceutical compositions are disclosed for the treatment of nicotine dependence or addiction, tobacco dependence or addiction, reduction of nicotine withdrawal symptoms or aiding in the cessation or lessening of tobacco use or substance abuse or other behavioral dependencies. The pharmaceutical compositions are comprised of a therapeutically effective combination of a nicotinic receptor partial agonist and a CB-1 receptor antagonist and a pharmaceutically acceptable carrier. The method of using these compounds is also disclosed.

### BACKGROUND OF THE INVENTION

The present invention relates to pharmaceutical compositions for the treatment of nicotine dependence or addiction in a mammal (e.g. human) comprising a nicotinic receptor partial agonist (NRPA) and a CB-1 receptor antagonist. The term NRPA refers to all chemical compounds that bind at neuronal nicotinic acetylcholine specific receptor sites in mammalian tissue and elicit a partial agonist response. A partial agonist response is defined here to mean a partial, or incomplete functional effect in a given functional assay. Additionally, a partial agonist will also exhibit some degree of antagonist activity by its ability to block the action of a full agonist (Feldman, R. S., Meyer, J. S. & Quenzer, L. F. Principles of Neuropsychopharmacology, 1997; Sinauer Assoc. Inc.). As used herein, the term “CB-1 Antagonists” refers to both full antagonists and partial antagonists, as well as inverse agonists of the G-protein coupled type 1 cannabinoid receptor. For a review of cannabinoid CB1 and CB2 receptor modulators, see Pertwee, R. G., “Cannabinoid Receptor Ligands: Clinical and Neuropharmacological Considerations, Relevant to Future Drug Discovery and Development,” Exp. Opin. Invest. Drugs, 9(7), 1553-1571 (2000). The present invention may be used to treat mammals (e.g. humans) for tobacco dependence or addiction and nicotine dependence or addiction; to palliate the effects of nicotine withdrawal, to enhance the outcomes of other smoking cessation therapies and to treat substance abuse and behavioral dependencies.

The invention also relates to aryl fused azapolycylic compounds that bind to neuronal nicotinic acetylcholine specific receptor sites and are useful in modulating cholinergic function and are referred to in WO 9818798 A1 (U.S. Pat. No. 6,235,734), WO 9935131-A1 (U.S. Pat. No. 6,410,550) and WO9955680-A1 (U.S. Pat. No. 6,462,035). The foregoing applications are owned in common with the present application and are incorporated herein by reference in their entireties.

The invention also relates to CB-1 receptor antagonists which include: (1) purine compounds such as those described in U.S. Provisional Application No. 60/421,874, filed on Oct. 28, 2002 and incorporated herein by reference; (2) pyrazolo[1,5-a}[1,3,5]triazine compounds such as those described in U.S. Provisional Application No. 60/445,728, filed on Feb. 6, 2003 and incorporated herein by reference; (3) pyrazolo[1,5-a]pyrimidine compounds such as those described in U.S. Provisional Application No. 60/446,450, filed on Feb. 10, 2003 and incorporated herein by reference; (4) 1,4- and 2,4-disubstituted imidazoles such as those disclosed in U.S. Provisional Application No. 60/419,621, filed on Oct. 18, 2002 and incorporated herein by reference; (5) 1-(1,5-diaryl-1H-pyrazol-3-yl)-2-(substituted amino)-ethanone compounds such as those described in U.S. Provisional Application No. 60/432,911, filed on Dec. 12, 2002 and incorporated herein by reference; (6) 1-(1,5-diaryl-1H-pyrazol-3-yl)-2-(substituted amino)-ethanol compounds such as those described in U.S. Provisional Application No. 60/432,911, filed on Dec. 12, 2002 and incorporated herein by reference; (7) 2-(1,5-diaryl-1H-pyrazol-3-yl)morpholine compounds such as those described in U.S. Provisional Application No. 60/432,911, filed on Dec. 12, 2002 and incorporated herein by reference; and (8) 1-(1,2-diaryl-1H-imidazol-4-yl)-2-(substituted amino)-ethanone compounds such as those described in U.S. Provisional Application No. 60/432,911, filed on Dec. 12, 2002 and incorporated herein by reference.

The NRPA compounds that bind to neuronal nicotinic receptor sites can be used in combination with a CB-1 receptor antagonist to treat addiction such as to nicotine or tobacco, alcohol dependence, cocaine addiction or tobacco or nicotine dependence independently of other psychiatric illness or other behavioral dependencies, eg. gambling.

Tobacco dependence represents the most important preventable cause of illness and death in our society, responsible for more than 400,000 deaths each year. Half of all smokers will die of diseases directly related to tobacco use, and many smokers will suffer significant morbidity.

People smoke because of the reinforcing effects of nicotine. Nicotine is a powerful psychoactive agent that activates the same brain pathways as cocaine and other psychostimulants, producing agent-associated tolerance and withdrawal effects.

Nicotine replacement therapies (NRTs) have been used for smoking cessation. These are available in the form of gum, the transdermal patch, and nasal inhaler. The gum Nicorette® (nicotine polacrilex) delivers nicotine through buccal absorption following chewing. There are also non-nicotine pharmacologic therapies for treating nicotine addiction.

### SUMMARY OF INVENTION

The present invention relates to a pharmaceutical composition for treating nicotine dependence or addiction, tobacco dependence or addiction, reducing nicotine withdrawal symptoms or aiding in the cessation or lessening of tobacco use or substance abuse or behavioral dependencies, comprising

(a) a nicotinic receptor partial agonist or a pharmaceutically acceptable salt thereof;

(b) a CB-1 receptor antagonist or pharmaceutically acceptable salt thereof; and

(c) a pharmaceutically acceptable carrier;

wherein the active agents “a” and “b” above are present in amounts that render the composition effective in treating nicotine dependence or addiction, tobacco dependence or addiction, reducing nicotine withdrawal symptoms or aiding in the cessation or lessening of tobacco use or substance abuse or behavioral dependencies. The therapeutically effective pharmaceutical combination is comprised of a nicotinic receptor partial agonist and a CB-1 receptor antagonist and a pharmaceutically acceptable carrier.

U.S. Provisional Application No. 60/421,874 describes CB-1 receptor antagonists purine compounds which are selected from: 1-[9-(4-chloro-phenyl)-8-(2-chlorophenyl)-9H-purin-6-yl]-3-ethylamino-azetidine-3-carboxylic acid amide; 1-[9-(4-chlorophenyl)-8-(2-chlorophenyl)-9H-purin-6-yl]-3-isopropylaminoazetidine-3-carboxylic acid amide; 1-{1-[9-(4-chlorophenyl)-8-(2-chlorophenyl)-9H-purin-6-yl]-4-phenyl piperidin-4-yl}-ethanone; {3-[9-(4-chlorophenyl)-8-(2,4-dichlorophenyl)-9H-purin-6-yl]-3-(1α,5α,6α)-azabicyclo[3.1.0]hex-6-yl}-dimethylamine; 6-(1-benzylpyrrolidin-3-yloxy)-9-(4-chlorophenyl)-8-(2,4-dichlorophenyl)-9H-purine; 9-(4-chlorophenyl)-6-(1-cyclohexylazetidin-3-yloxy)-8-(2,4-dichlorophenyl)-9H-purine; 6-tert-butoxy-9-(4-chlorophenyl)-8-(2,4-dichlorophenyl)-9H-purine; 9-(4-chlorophenyl)-8-(2,4-dichlorophenyl)-6-isopropoxy-9H-purine; 1-[9-(4-chlorophenyl)-8-(2,4-dichlorophenyl)-9H-purin-6-yl]-4-propylaminopiperidine-4-carboxylic acid amide; 1-[9-(4-chlorophenyl)-8-(2-fluorophenyl)-9H-purin-6-yl]-4-propylaminopiperidine-4-carboxylic acid amide; 1-[9-(4-chlorophenyl)-8-(2-chlorophenyl)-9H-purin-6-yl]-4-propylaminopiperidine-4-carboxylic acid amide; 1-[9-(4-chlorophenyl)-8-(2-fluorophenyl)-2-methyl-9H-purin-6-yl]-4-isopropylaminopiperidine-4-carboxylic acid amide; 1-[9-(4-chlorophenyl)-8-(2-chlorophenyl)-9H-purin-6-yl]-4-pyrrolidin-1-yl-piperidine-4-carboxylic acid amide; 1-[9-(4-chlorophenyl)-8-(2-chlorophenyl)-9H-purin-6-yl]-4-ethylamino-piperidine-4-carboxylic acid amide; 1-[9-(4-chlorophenyl)-8-(2-chlorophenyl)-9H-purin-6-yl]-4-isopropylaminopiperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2,4-dichlorophenyl)-9H-purin-6-yl]-4-methylaminopiperidine-4-carboxylic acid amide; 1-[9-(4-chlorophenyl)-8-(2-fluorophenyl)-9H-purin-6-yl]-4-isopropylaminopiperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 9-[9-(4-chlorophenyl)-8-(2-chlorophenyl)-9H-purin-6-yl]-1-methyl-4-oxa-1,9-diazaspiro[5.5]undecan-2-one; 8-[9-(4-chlorophenyl)-8-(2,4-dichlorophenyl)-9H-purin-6-yl]-1-isopropyl-1,3,8-triazaspiro[4.5]decan-4-one; 1-[9-(4-chlorophenyl)-8-(2-chlorophenyl)-9H-purin-6-yl]-4-(4-fluorophenyl)-piperidin-4-ol; 1-[9-(4-chlorophenyl)-8-(2-chlorophenyl)-9H-purin-6-yl]-4-phenylpiperidin-4-ol; 4-benzyl-1-[9-(4-chlorophenyl)-8-(2-chlorophenyl)-9H-purin-6-yl]-piperidin-4-ol; 4-[9-(4-chlorophenyl)-8-(2-chlorophenyl)-9H-purin-6-yl]-piperazine-2-carboxylic acid methylamide; 9-(4-chlorophenyl)-8-(2,4-dichlorophenyl)-6-(4-pyridin-2-yl-piperazin-1-yl)-9H-purine; and 9-(4-chlorophenyl)-8-(2,4-dichlorophenyl)-6-(4-pyrimidin-2-yl-piperazin-1-yl)-9H-purine; 1-[9-(4-chlorophenyl)-8-(2-fluorophenyl)-9H-purin-6-yl]-4-isopropylamino-piperidine-4-carboxylic acid amide; 1-[9-(4-chlorophenyl)-8-(2-chlorophenyl)-9H-purin-6-yl]-4-isopropylamino-piperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2-chlorophenyl)-9H-purin-6-yl]-4-ethylamino-piperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 4-amino-1-[9-(4-chloro-phenyl)-8-(2-chlorophenyl)-9H-purin-6-yl]-piperidine-4-carboxylic acid amide; and 1-[9-(4-chlorophenyl)-8-(2-chlorophenyl)-9H-purin-6-yl]-4-ethylaminopiperidine-4-carboxylic acid amide; and a pharmaceutically acceptable salt thereof or a solvate or hydrate of said compound or said salt.

U.S. Provisional Application No. 60/445,728 describes CB-1 receptor antagonist pyrazolo[1,5-a}[1,3,5]triazine compounds selected from: 7-(2-chlorophenyl)-8-(4-chlorophenyl)-2-methyl-4-(4-methylpiperazin-1-yl)-pyrazolo[1,5-a][1,3,5]triazine; 7-(2-chlorophenyl)-8-(4-chlorophenyl)-2-methyl-4-(4-pyrimidin-2-ylpiperazin-1-yl)-pyrazolo[1,5-a][1,3,5]triazine; 7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and 7-(2-chlorophenyl)-8-(4-chlorophenyl)-2-methyl-4-[4-(propane-2-sulfonyl)-piperazin-1-yl]-pyrazolo[1,5-a][1,3,5]triazine; 1-[7-(2-chlorophenyl)-8-(4-chlorophenyl)-2-methylpyrazolo[1,5-a][1,3,5]triazin-4-yl]-4-methylaminopiperidine-4-carboxylic acid amide; 1-[7-(2-chlorophenyl)-8-(4-fluorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-isopropylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; 1-[7-(2-chlorophenyl)-8-(4-chlorophenyl)-2-methylpyrazolo[1,5-a][1,3,5]triazin-4-yl]-3-methylaminoazetidine-3-carboxylic acid amide; and 1-[7-(2-chlorophenyl)-8-(4-chlorophenyl)-2-methylpyrazolo[1,5-a][1,3,5]triazin-4-yl]-3-dimethylaminoazetidine-3-carboxylic acid amide; 1-{1-[7-(2-chlorophenyl)-8-(4-chlorophenyl)-2-methylpyrazolo[1,5-a][1,3,5]triazin-4-yl]-4-phenylpiperidin-4-yl}-ethanone; 3-[7-(2-chlorophenyl)-8-(4-chlorophenyl)-2-methylpyrazolo[1,5-a][1,3,5]triazin-4-yl]-3-azabicyclo[3.1.0]hex-6-ylamine; 1-[7-(2-chlorophenyl)-8-(4-chlorophenyl)-2-methylpyrazolo[1,5-a][1,3,5]triazin-4-yl]-4-(4-fluorophenyl)-piperidin-4-ol; 4-benzyl-1-[7-(2-chlorophenyl)-8-(4-chlorophenyl)-2-methylpyrazolo[1,5-a][1,3,5]triazin-4-yl]-piperidin-4-ol; 2-[7-(2-chlorophenyl)-8-(4-chlorophenyl)-2-methylpyrazolo[1,5-a][1,3,5]triazin-4-yl]-5-methyl-2,5,7-triazaspiro[3.4]octan-8-one; 2-[7-(2-chlorophenyl)-8-(4-chlorophenyl)-2-methylpyrazolo[1,5-a][1,3,5]triazin-4-yl]-2,5,7-triazaspiro[3.4]octan-8-one; 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; 2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one; 4-(1-benzylpyrrolidin-3-yloxy)-7-(2-chlorophenyl)-8-(4-chlorophenyl)-2-methylpyrazolo[1,5-a][1,3,5]triazine; 7-(2-chlorophenyl)-8-(4-chlorophenyl)-4-(1-cyclohexylazetidin-3-yloxy)-2-methylpyrazolo[1,5-a][1,3,5]triazine; 7-(2-chlorophenyl)-8-(4-chlorophenyl)-4-isopropoxy-2-methylpyrazolo[1,5-a][1,3,5]triazine; and 4-tert-butoxy-7-(2-chlorophenyl)-8-(4-chlorophenyl)-2-methylpyrazolo[1,5-a][1,3,5]triazine; butyl-[7-(2-chlorophenyl)-8-(4-chlorophenyl)-2-methylpyrazolo[1,5-a][1,3,5]triazin-4-yl]-amine; 7-(2-chlorophenyl)-8-(4-chlorophenyl)-2-methyl-4-piperidin-1-yl-pyrazolo[1,5-a][1,3,5]triazine; [7-(2-chlorophenyl)-8-(4-chlorophenyl)-2-methylpyrazolo[1,5-a][1,3,5]triazin-4-yl]-[2-(4-fluorophenyl)-ethyl]-amine; 7-(2-chlorophenyl)-8-(4-chlorophenyl)-2-methyl-4-morpholin-4-yl-pyrazolo[1,5-a][1,3,5]triazine; and [7-(2-chlorophenyl)-8-(4-chlorophenyl)-2-methylpyrazolo[1,5-a][1,3,5]triazin-4-yl]-(2-morpholin-4-yl-ethyl)-amine; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; and 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and a pharmaceutically acceptable salt thereof or a solvate or hydrate of said compound or said salt.

U.S. Provisional Application No. 60/446,450 describes CB-1 receptor antogonist pyrazolo[1,5-a]pyrimidine compounds selected from: 3-(4-chlorophenyl)-2-(2-chlorophenyl)-7-(4-methyl-piperazin-1-yl)-pyrazolo[1,5-a]pyrimidine; 3-(4-chlorophenyl)-2-(2-chlorophenyl)-7-(4-pyrimidin-2-yl-piperazin-1-yl)-pyrazolo[1,5-a]pyrimidine; 3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and 3-(4-chlorophenyl)-2-(2-chlorophenyl)-7-[4-(propane-2-sulfonyl)-piperazin-1-yl]-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; 1-[3-(4-chlorophenyl)-2-(2-chlorophenyl)-pyrazolo[1,5-a]pyrimidin-7-yl]-3-ethylaminoazetidine-3-carboxylic acid amide; 3-amino-1-[3-(4-chlorophenyl)-2-(2-chlorophenyl)-pyrazolo[1,5-a]pyrimidin-7-yl]-azetidine-3-carboxylic acid amide; 1-[3-(4-chlorophenyl)-2-(2-chlorophenyl)-6-methylpyrazolo[1,5-a]pyrimidin-7-yl]-3-ethylaminoazetidine-3-carboxylic acid amide; 1-[3-(4-chlorophenyl)-2-(2-chlorophenyl)-pyrazolo[1,5-a]pyrimidin-7-yl]-3-isopropylaminoazetidine-3-carboxylic acid amide; 1-[3-(4-chlorophenyl)-2-(2-chlorophenyl)-5,6-dimethylpyrazolo[1,5-a]pyrimidin-7-yl]-3-ethylamino-azetidine-3-carboxylic acid amide; 1-[3-(4-chlorophenyl)-2-(2-chlorophenyl)-pyrazolo[1,5-a]pyrimidin-7-yl]-3-methylaminoazetidine-3-carboxylic acid amide; 1-[3-(4-chlorophenyl)-2-(2-chlorophenyl)-5-methylpyrazolo[1,5-a]pyrimidin-7-yl]-3-ethylaminoazetidine-3-carboxylic acid amide; 1-{1-[3-(4-chlorophenyl)-2-(2-chlorophenyl)-pyrazolo[1,5-a]pyrimidin-7-yl]-4-phenylpiperidin-4-yl}-ethanone; 3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine; 1-[3-(4-chlorophenyl)-2-(2-chlorophenyl)-pyrazolo[1,5-a]pyrimidin-7-yl]-4-(4-fluorophenyl)-piperidin-4-ol; 4-benzyl-1-[3-(4-chlorophenyl)-2-(2-chlorophenyl)-pyrazolo[1,5-a]pyrimidin-7-yl]-piperidin-4-ol; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-2,5,7-triazaspiro[3.4]octan-8-one; 8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-5-methyl-2,5,7-triazaspiro[3.4]octan-8-one; 7-(1-benzylpyrrolidin-3-yloxy)-3-(4-chlorophenyl)-2-(2-chlorophenyl)-pyrazolo[1,5-a]pyrimidine; and 3-(4-chlorophenyl)-2-(2-chlorophenyl)-7-(1-cyclohexylazetidin-3-yloxy)-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; and 1-[3-(4-chlorophenyl)-2-(2-chlorophenyl)-pyrazolo[1,5-a]pyrimidin-7-yl]-3-ethylaminoazetidine-3-carboxylic acid amide; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; and a pharmaceutically acceptable salt thereof or a solvate or hydrate of said compound or said salt.

U.S. Provisional Application No. 60/419,621 describes CB-1 receptor antagonist 1,4- and 2,4-disubstituted imidazoles selected from: 5-(4-chloro-phenyl)-3-(5-cyclohexyl-1H-imidazol-2-yl)-1-(2,4-dichloro-phenyl)-4-methyl-1H-pyrazole; 5-(4-chloro-phenyl)-3-(2-cyclohexyl-3H-imidazol-4-yl)-1-(2,4-dichloro-phenyl)-4-methyl-1H-pyrazole; 5-(4-chloro-phenyl)-1-(2,4-dichloro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-4-methyl-3-[1-(1-phenyl-ethyl)-1H-imidazol-4-yl]-1H-pyrazole; 5-(4-chloro-phenyl)-1-(2-fluoro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole: 5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and 1-(2-chloro-phenyl)-5-(4-chloro-phenyl)-4-methyl-3-[1-(1-methyl-1-phenyl-ethyl)-1H-imidazol-4-yl]-1H-pyrazole; and a pharmaceutically acceptable salt thereof or a solvate or hydrate of the compound or the salt.

U.S. Provisional Application No. 60/432,911 describes CB-1 receptor antagonist 1-(1,5-diaryl-1H-pyrazol-3-yl)-2-(substituted amino)-ethanol compounds selected from: 1-[5-(4-chloro-phenyl)-1-(2-chloro-phenyl)-4-methyl-1H-pyrazol-3-yl]-2-piperidin-1-yl-ethanone; 1-[5-(4-chloro-phenyl)-1-(2-chloro-phenyl)-4-methyl-1H-pyrazol-3-yl]-2-morpholin-4-yl-ethanone; 1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone; 1-[5-(4-chloro-phenyl)-1-(2-chloro-phenyl)-4-methyl-1H-pyrazol-3-yl]-2-[4-(1-methyl-cyclopropanecarbonyl)-piperazin-1-yl]-ethanone; N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide; 1-[5-(4-chloro-phenyl)-1-(2-fluoro-phenyl)-4-methyl-1H-pyrazol-3-yl]-2-morpholin-4-yl-ethanone; 1-[5-(4-chloro-phenyl)-1-(2-fluoro-phenyl)-4-methyl-1H-pyrazol-3-yl]-2-piperidin-1-yl-ethanone; 1-[5-(4-chloro-phenyl)-1-(2-fluoro-phenyl)-4-methyl-1H-pyrazol-3-yl]-2-(4-trifluoroacetyl-piperazin-1-yl)-ethanone; 1-[1-(2-chloro-phenyl)-5-(4-chloro-phenyl)-4-methyl-1H-pyrazol-3-yl]-2-pyrrolidin-1-yl-ethanone; 1-[1-(2-chloro-phenyl)-5-(4-chloro-phenyl)-4-methyl-1H-pyrazol-3-yl]-2-[1,4]oxazepan-4-yl-ethanone; and 1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and a pharmaceutically acceptable salt thereof, or a solvate or hydrate of the compound or the salt.

U.S. Provisional Application No. 60/432,911 describes CB-1 receptor antagonist 1-(1,5-diaryl-1H-pyrazol-3-yl)-2-(substituted amino)-ethanol compounds selected from: 2-(benzyl-isopropyl-amino)-1-[1-(2-chloro-phenyl)-5-(4-chloro-phenyl)-4-methyl-1H-pyrazol-3-yl]-ethanol; 1-[5-(4-chloro-phenyl)-1-(2-chloro-phenyl)-4-methyl-1H-pyrazol-3-yl]-2-(3,5-dimethyl-piperidin-1-yl)-ethanol; 1-{2-[1-(2-chloro-phenyl)-5-(4-chloro-phenyl)-4-methyl-1H-pyrazol-3-yl]-2-hydroxy-ethyl}-4-isopropylamino-piperidine-4-carboxylic acid amide; 1-[5-(4-chloro-phenyl)-1-(2-chloro-phenyl)-4-methyl-1H-pyrazol-3-yl]-2-(3,3-dimethyl-piperidin-1-yl)-ethanol; 1-[5-(4-chloro-phenyl)-1-(2-chloro-phenyl)-4-methyl-1H-pyrazol-3-yl]-2-piperidin-1-yl-ethanol; and 1-[5-(4-chloro-phenyl)-1-(2-chloro-phenyl)-4-methyl-1H-pyrazol-3-yl]-2-morpholin-4-yl-ethanol; and a pharmaceutically acceptable salt thereof, or a solvate or hydrate of the compound or the salt.

U.S. Provisional Application No. 60/432,911 describes CB-1 receptor antagonist 1-(1,2-diaryl-1H-imidazol-4-yl)-2-(substituted amino)-ethanone compounds selected from: 1-[1-(4-chloro-phenyl)-2-(2,4-dichloro-phenyl)-5-methyl-1H-imidazol-4-yl]-2-piperidin-1-yl-ethanone and 1-[1-(4-chloro-phenyl)-2-(2,4-dichloro-phenyl)-5-methyl-1H-imidazol-4-yl]-2-morpholin-4-yl-ethanone; and a pharmaceutically acceptable salt thereof, or a solvate or hydrate of the compound or the salt.

In another more specific embodiment of this invention, the nicotinic receptor partial agonist is selected from:

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1,5]diazocin-8-one;

9-methyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

9-vinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2-a](1,5]diazocin-8-one;

3-benzyl-9-bromo-1,2,3,4,5,6-hexahydro-1,5-methano-pyrido[1,2-a][1,5]diazocin-8-one;

3-benzyl-9-chloro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-ethynyl-1,2,3,4,5,6-hexahydro-1,5-methano-pyrido[1,2a][1,5]diazocin-8-one;

9-(2-propenyl)-1,2,3,4,5,6-hexahydro-1,5-methano-pyrido[1,2a][1,5]diazocin-8-one;

9-(2-propyl)-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

9-(4-fluorophenyl)-1,2,3,4,5,6-hexahydro-1,5-methano-pyrido[1,2a][1,5]diazocin-8-one;

9-(3-fluorophenyl)-1,2,3,4,5,6-hexahydro-1,5-methano-pyrido[1,2a][1,5]diazocin-8-one;

9-(3,5-difluorophenyl)-1,2,3,4,5,6-hexahydro-1,5-methano-pyrido[1,2a][1,5]diazocin-8-one;

9-(2,4-difluorophenyl)-1,2,3,4,5,6-hexahydro-1,5-methano-pyrido[1,2a][1,5]diazocin-8-one;

9-(2,5-difluorophenyl)-1,2,3,4,5,6-hexahydro-1,5-methano-pyrido[1,2a][1,5]diazocin-8-one;

6-methyl-5-oxo-6,13-diazatetracyclo[9.3.1.0^{2,10}.0^{4,8}]pentadeca-2(10),3,8-triene;

5-oxo-6,13-diazatetracyclo[9.3.1.0^{2,10}.0^{4,8}]pentadeca-2(10),3,8-triene;

6-oxo-5,7,13-triazatetracyclo[9.3.1.0^{2,10}.0^{4,8}]pentadeca-2(10),3,8-triene;

4,5-difluoro-10-aza-tricyclo[6.3.1.0^{2,7}]dodeca-2(7),3,5-triene;

5-fluoro-10-aza-tricyclo[6.3.1.0^{2,7}]dodeca-2(7),3,5-triene-4-carbonitrile;

4-ethynyl-5-fluoro-10-aza-tricyclo[6.3.1.0^{2,7}]dodeca-2(7),3,5-triene;

5-ethynyl-10-aza-tricyclo[6.3.1.0^{2,7}]dodeca-2(7),3,5-triene-4-carbonitrile;

6-methyl-5-thia-5-dioxa-6,13-diazatetracyclo[9.3.1.0^{2,10}.0^{4,8}]pentadeca-2(10),3,8-triene;

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

7-methyl-5,7,13-triazatetracyclo[9.3.1.0^{2,10}.0^{4,8}]pentadeca-2(10),3,5,8-tetraene;

6-methyl-5,7,13-triazatetracyclo[9.3.1.0^{2,10}.0^{4,8}]pentadeca-2(10),3,5,8-tetraene;

6,7-dimethyl-5,7,13-triazatetracyclo[9.3.1.0^{2,10}.0^{4,8}]pentadeca-2(10),3,5,8-tetraene;

6-methyl-7-phenyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

b 5,8,14-triazatetracyclo[10.3.1.0^{2,11}.0^{4,9}]hexadeca-2(11),3,5,7,9-pentaene;

14-methyl-5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

4-chloro-10-azatricyclo[6.3.1.0^{2,7}]dodeca-2(7),3,5-triene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4,5-dichloro-10-azatricyclo[6.3.1.0^{2,7}]dodeca-2(7),3,5-triene;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

5,7,14-triazatetracyclo[10.3.1.0^{2,10}.0^{4,8}]hexadeca-2(10),3,5,8-tetraene;

5,6-dimethyl-5,7,14-triazatetracyclo[10.3.1.0^{2,10}.0^{4,8}]hexadeca-2(10),3,6,8-tetraene;

5-methyl-5,7,14-triazatetracyclo[10.3.1.0^{2,10}.0^{4,8}]hexadeca-2(10),3,6,8-tetraene;

6-(trifluoromethyl)-7-thia-5,14-diazatetracyclo[10.3.1.0^{2,10}.0^{4,8}]hexadeca-2(10),3,5,8-tetraene;

5,8,15-triazatetracyclo[11.3.1.0^{2,11}.0^{4,9}]heptadeca-2(11),3,5,7,9-pentaene;

7-methyl-5,8,15-triazatetracyclo[11.3.1.0^{2,11}.0^{4,9}]heptadeca-2(11),3,5,7,9-pentaene;

6-methyl-5,8,15-triazatetracyclo[11.3.1.0^{2,11}.0^{4,9}]heptadeca-2(11),3,5,7,9-pentaene;

6,7-dimethyl-5,8,15-triazatetracyclo[11.3.1.0^{2,11}.0^{4,9}]heptadeca-2(11),3,5,7,9-pentaene;

7-oxa-5,14-diazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

5-methyl-7-oxa-6,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

7-methyl-5-oxa-6,14-diazatetracyclo[10.3.1.0^{2,10}.0^{4,8}]hexadeca-2(10),3,6,8-tetraene;

4,5-difluoro-11-azatricyclo[7.3.1.0^{2,7}]trideca-2(7),3,5-triene;

4-chloro-5-fluoro-11-azatricyclo[7.3.1.0^{2,7}]trideca-2(7),3,5-triene;

5-chloro-4-fluoro-11-azatricyclo[7.3.1.0^{2,7}]trideca-2(7),3,5-triene;

4-(1-ethynyl)-5-fluoro-11-azatricyclo[7.3.1.0^{2,7}]trideca-2(7),3,5-triene;

5-(1-ethynyl)-4-fluoro-11-azatricyclo[7.3.1.0^{2,7}]trideca-2(7),3,5-triene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene;

11-aza-tricyclo[7.3.1.0^{2,7}]trideca-2(7),3,5-trien-5-ol;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

5-fluoro-11-aza-tricyclo[7.3.1.0^{2,7}]trideca-2(7),3,5-triene; and

6-hydroxy-5-methoxy-11-aza-tricyclo[7.3.1.0^{2,7}]trideca-2(7),3,5-triene and

their pharmaceutically acceptable salts and their optical isomers.

Preferably, the nicotinic receptor partial agonist is selected from

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

6-methyl-5-thia-5-dioxa-6,13-diazatetracyclo[9.3.1.1.0^{2,10}.0^{4,8}]pentadeca-2(10),3,8-triene;

4-fluoro-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5triene;

4-nitro-10-azatricyclo[6.3.1.0^{2,7}]dodeca-2(7),3,5-triene;

6-dimethyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,11}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

6-fluoro-11-aza-tricyclo[7.3.1.0^{2,7}]trideca-2(7),3,5-triene; and

11-aza-tricyclo[7.3.1.0^{2,7}]trideca-2(7),3,5-trien-5-ol and

their pharmaceutically acceptable salts and their optical isomers.

The present invention also relates to a method of treating nicotine dependence or addiction, tobacco dependence or addiction, reducing nicotine withdrawal symptoms or aiding in the cessation or lessening of tobacco use or substance abuse or behavioral dependencies, comprising

(a) a nicotinic receptor partial agonist or a pharmaceutically acceptable salt thereof; and

(b) a CB-1 receptor antagonist or pharmaceutically acceptable salt thereof;

wherein the active agents (a) and (b) above are present in amounts that render the composition effective in treating nicotine dependence or addiction, tobacco dependence or addiction, reducing nicotine withdrawal symptoms or aiding in the cessation or lessening of tobacco use or substance abuse or behavioral dependencies.

The nicotinic receptor partial agonist and the a CB-1 receptor antagonist are present in amounts that render the composition effective in the treatment of tobacco or nicotine addiction, nicotine withdrawal symptoms, substance abuse or other behavioral dependencies.

In a more specific embodiment of the invention, the CB-1 receptor antagonists, are listed herein above.

In another more specific embodiment of this invention the nicotinic receptor partial agonist is selected from:

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1,5]diazocin-8-one;

9-methyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

9-vinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2-a][1,5]diazocin-8-one;

3-benzyl-9-bromo-1,2,3,4,5,6-hexahydro-1,5-methano-pyrido[1,2-a][1,5]diazocin-8-one;

3-benzyl-9-chloro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-ethynyl-1,2,3,4,5,6-hexahydro-1,5-methano-pyrido[1,2a][1,5]diazocin-8-one;

9-(2-propenyl)-1,2,3,4,5,6-hexahydro-1,5-methano-pyrido[1,2a][1,5]diazocin-8-one;

9-(2-propyl)-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

9-(4-fluorophenyl)-1,2,3,4,5,6-hexahydro-1,5-methano-pyrido[1,2a][1,5]diazocin-8-one;

9-(3-fluorophenyl)-1,2,3,4,5,6-hexahydro-1,5-methano-pyrido[1,2a][1,5]diazocin-8-one;

9-(3,5-difluorophenyl)-1,2,3,4,5,6-hexahydro-1,5-methano-pyrido[1,2a][1,5]diazocin-8-one;

9-(2,4-difluorophenyl)-1,2,3,4,5,6-hexahydro-1,5-methano-pyrido[1,2a][1,5]diazocin-8-one;

9-(2,5-difluorophenyl)-1,2,3,4,5,6-hexahydro-1,5-methano-pyrido[1,2a][1,5]diazocin-8-one;

6-methyl-5-oxo-6,13-diazatetracyclo[9.3.1.0^{2,10}.0^{4,8}]pentadeca-2(10),3,8-triene;

5-oxo-6,13-diazatetracyclo[9.3.1.0^{2,10}.0^{4,8}]pentadeca-2(10),3,8-triene;

6-oxo-5,7,13-triazatetracyclo[9.3.1.0^{2,10}.0^{4,8}]pentadeca-2(10),3,8-triene;

4,5-difluoro-10-aza-tricyclo[6.3.1.0^{2,7}]dodeca-2(7),3,5-triene;

5-fluoro-10-aza-tricyclo[6.3.1.0^{2,7}]dodeca-2(7),3,5-triene-4-carbonitrile;

4-ethynyl-5-fluoro-10-aza-tricyclo[6.3.1.0^{2,7}]dodeca-2(7),3,5-triene;

5-ethynyl-10-aza-tricyclo[6.3.1.0^{2,7}]dodeca-2(7),3,5-triene-4-carbonitrile;

6-methyl-5-thia-5-dioxa-6,13-diazatetracyclo[9.3.1.0^{2,10}.0^{4,8}]pentadeca-2(10),3,8-triene;

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

7-methyl-5,7,13-triazatetracyclo[9.3.1.0^{2,10}.0^{4,8}]pentadeca-2(10),3,5,8-tetraene;

6-methyl-5,7,13-triazatetracyclo[9.3.1.0^{2,10}.0^{4,8}]pentadeca-2(10),3,5,8-tetraene;

6,7-dimethyl-5,7,13-triazatetracyclo[9.3.1.0^{2,10}.0^{4,8}]pentadeca-2(10),3,5,8-tetraene;

6-methyl-7-phenyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

14-methyl-5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

4-chloro-10-azatricyclo[6.3.1.0^{2,7}]dodeca-2(7),3,5-triene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4,5-dichloro-10-azatricyclo[6.3.1.0^{2,7}]dodeca-2(7),3,5-triene;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

5,7,14-triazatetracyclo[10.3.1.0^{2,10}.0^{4,8}]hexadeca-2(10),3,5,8-tetraene;

5,6-dimethyl-5,7,14-triazatetracyclo[10.3.1.0^{2,10}.0^{4,8}]hexadeca-2(10),3,6,8-tetraene;

5-methyl-5,7,14-triazatetracyclo[10.3.1.0^{2,10}.0^{4,8}]hexadeca-2(10),3,6,8-tetraene;

6-(trifluoromethyl)-7-thia-5,14-diazatetracyclo[10.3.1.0^{2,10}.0^{4,8}]hexadeca-2(10),3,5,8-tetraene;

5,8,15-triazatetracyclo[11.3.1.0^{2,11}.0^{4,9}]heptadeca-2(11),3,5,7,9-pentaene;

7-methyl-5,8,15-triazatetracyclo[11.3.1.0^{2,11}.0^{4,9}]heptadeca-2 (11),3,5,7,9-pentaene;

7-methyl-5,8,5-triazatetracyclo[11.3.1.0^{2,10}.0^{4,9}]heptadeca-2(11),3,5,7,9-pentaene;

6-methyl-5,8,15-triazatetracyclo[11.3.1.0^{2,11}.0^{4,9}]heptadeca-2(11),3,5,7,9-pentaene;

6,7-dimethyl-5,8,15-triazatetracyclo[11.3.1.0^{2,11}.0^{4,9}]heptadeca-2(11),3,5,7,9-pentaene;

7-oxa-5,14-diazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2 (10),3,5,8-tetraene;

5-methyl-7-oxa-6,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

7-methyl-5-oxa-6,14-diazatetracyclo[10.3.1.0^{2,10}.0^{4,8}]hexadeca-2(10),3,6,8-tetraene;

4,5-difluoro-11-azatricyclo[7.3.1.0^{2,7}]trideca-2(7),3,5-triene;

4-chloro-5-fluoro-11-azatricyclo[7.3.1.0^{2,7}]trideca-2(7),3,5-triene;

5-chloro-4-fluoro-11-azatricyclo[7.3.1.0^{2,7}]trideca-2(7),3,5-triene;

4-(1-ethynyl)-5-fluoro-11-azatricyclo[7.3.1.0^{2,7}]trideca-2(7),3,5-triene;

5-(1-ethynyl)-4-fluoro-11-azatricyclo[7.3.1.0^{2,7}]trideca-2(7),3,5-triene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene,

11-aza-tricyclo[7.3.1.0^{2,7}]trideca-2(7),3,5-trien-5-ol;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

5-fluoro-11-aza-tricyclo[7.3.1.0^{2,7}]trideca-2(7),3,5-triene; and

6-hydroxy-5-methoxy-11-aza-tricyclo[7.3.1.0^{2,7}]trideca-2(7),3,5-triene and

their pharmaceutically acceptable salts and their optical isomers.

Preferably, the nicotinic receptor partial agonist is selected from:

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl)-1,2,3,4,5,6-hexahydro-1,5-methano-pyrido[1,2a][1,5]diazocin-8-one;

6-methyl-5-thia-5-dioxa-6,13-diazatetracyclo[9.3.1.0^{2,10}. 0^{4,8}]pentadeca-2(10),3,8-triene;

4-fluoro-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

6-methyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

6-fluoro-11-aza-tricyclo[7.3.1.0^{2,7}]trideca-2(7),3,5-triene; and

11-aza-tricyclo[7.3.1.0^{2,7}]trideca-2(7),3,5-trien-5-ol and the pharmaceutically acceptable salts and optical isomers of the foregoing compounds.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above.

The term “substance abuse”, as used herein, for example in “drug addiction” and “alcohol addiction”, unless otherwise indicated, refers to a maladaptive use of a substance, which may be either with physiological dependence or without. The term “substance abuse” thus includes both substance abuse (e.g. alcohol, amphetamine, cocaine or an opioid, for example morphine, opium, or heroine, abuse) and substance dependence (e.g. alcohol, amphetamine, cocaine or an opioid, for example morphine, opium, or heroine dependence). The maladaptive pattern of substance use may manifest itself in recurrent and significant adverse consequences related to the repeated use of the substance. The recurrent substance use may result in a failure to fulfill major role obligations at work, school, or home. The maladaptive use of a substance may involve continued use of the substance despite persistent or recurrent social or interpersonal problems caused or exacerbated by the effects of the substance (e.g., arguments with spouse, physical fights). The maladaptive pattern of substance use may involve clinically significant impairment or distress, for example manifested by tolerance for the substance, withdrawal symptoms, unsuccessful efforts to cut down or control the substance use, and/or taking larger amounts of the substance and/or taking amounts of the substance over a longer period than was intended. Substances to which an addiction may be formed include, but are not limited to, the drugs recited above (including alcohol), as well as others, for example benzodiazepines such as Valium®.

Behavioral dependencies as used here means enduring or persistent patterns of behavior which deviates markedly from the expectations of an individual's culture, is pervasive and inflexible, is stable over time, and leads to distress or impairment, and can include either Axis I or Axis II diagnoses (1994; DSM-IV, American Psychiatric Association). Such diagnoses may include, but are not limited to, substance abuse (nicotine, alcohol, narcotics, inhalants), gambling, eating disorders, and impulse control disorders.

The chemist of ordinary skill will recognize that certain compounds of this invention will contain one or more atoms which may be in a particular stereochemical or geometric configuration, giving rise to stereoisomers and configurational isomers. All such isomers and mixture thereof are included in this invention. Hydrates of the compounds of this invention are also included.

The chemist of ordinary skill will recognize that certain combinations of heteroatom-containing substituent listed in this invention define compounds which will be less stable under physiological conditions (e.g., those containing acetal or animal linkages). According, such compounds are less preferred.

### DETAILED DESCRIPTION OF THE INVENTION

In combination with the NRPA, the invention includes a CB-1 receptor antagonist and a pharmaceutically acceptable salt thereof.

The particular NRPA compounds listed above, which can be employed in the methods and pharmaceutical compositions of this invention, can be made by processes known in the chemical arts, for example by the methods described in WO 9818798 A1 (U.S. Pat. No. 6,235,734), WO 9935131-A1 (U.S. Pat. No. 6,410,550) and WO9955680-A1 (U.S. Pat. No. 6,462,035). Some of the preparation methods useful for making the compounds of this invention may require protection of remote functionality (i.e., primary amine, secondary amine, carboxyl). The need for such protection will vary depending on the nature of the remote functionality and the conditions of the preparation methods. The need for such protection is readily determined by one skilled in the art, and is described in examples carefully described in the above cited applications. The starting materials and reagents for the NRPA compounds employed in this invention are also readily available or can be easily synthesized by those skilled in the art using conventional methods of organic synthesis. Some of the compounds used herein are related to, or are derived from compounds found in nature and accordingly many such compounds are commercially available or are reported in the literature or are easily prepared from other commonly available substances by methods which are reported in the literature.

Some of the NRPA compounds employed in this invention are ionizable at physiological conditions. Thus, for example some of the compounds of this invention are acidic and they form a salt with a pharmaceutically acceptable cation. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the NRPA compounds employed in this invention are basic, and they form a salt with a pharmaceutically acceptable anion. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, when the NRPA compounds employed in this invention form hydrates or solvates they are also within the scope of the invention.

Some of the compounds of this invention are chiral, and as such are subject to preparation via chiral synthetic routes, or separable by conventional resolution or chromatographic means. All optical forms of the compounds of this invention are within the scope of the invention.

The utility of the NRPA compounds employed in the present invention as medicinal agents in the treatment of nicotine dependence (such as tobacco dependence or addiction) in mammals (e.g. humans) is demonstrated by the activity of the compounds of this invention in conventional assays and, in particular the assays described below. These include neuronal nicotinic receptor binding, dopamine turnover. Such assays also provide a means whereby the activities of the compounds of this invention can be compared between themselves and with the activities of other known compounds. The results of these comparisons are useful for determining dosage levels in mammals, including humans, for the treatment of such diseases.

### Biological Assays

### Procedures

Receptor binding assay: The effectiveness of the active compounds in suppressing nicotine binding to specific receptor sites is determined by the following procedure which is a modification of the methods of Lippiello, P. M. and Fernandes, K. G. (in The Binding of L-[³H]Nicotine To A Single Class of High-Affinity Sites in Rat Brain Membranes, Molecular Pharm., 29, 448-54, (1986)) and Anderson, D. J. and Arneric, S. P. (in Nicotinic Receptor Binding of ³H-Cystisine, ³H-Nicotine and ³H-Methylcarmbamylcholine In Rat Brain, European J. Pharm., 253, 261-67 (1994)). Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum. The rats were killed by decapitation. Brains were removed immediately following decapitation. Membranes were prepared from brain tissue according to the methods of Lippiello and Fernandez (Molec Pharmacol, 29, 448-454, (1986) with some modifications. Whole brains were removed, rinsed with ice-cold buffer, and homogenized at 0° in 10 volumes of buffer (w/v) using a Brinkmann Polytron™, setting 6, for 30 seconds. The buffer consisted of 50 mM Tris HCl at a pH of 7.5 at room temperature. The homogenate was sedimented by centrifugation (10 minutes; 50,000×g; 0 to 4° C). The supernatant was poured off and the membranes were gently resuspended with the Polytron and centrifuged again (10 minutes; 50,000×g; 0 to 4° C. After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and has a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [3H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0 to 4° C. in an iced shaking water bath. Incubations were terminated by rapid filtration under vacuum through Whatman GF/B™ glass fiber filters using a Brandel™ multi-manifold tissue harvester. Following the initial filtration of the assay mixture, filters were washed two times with ice-cold assay buffer (5 mL each). The filters were then placed in counting vials and mixed vigorously with 20 ml of Ready Safe™ (Beckman) before quantification of radioactivity. Samples were counted in a LKB Wallach Rackbeta™ liquid scintillation counter at 40-50% efficiency. All determinations were in triplicate.

Calculations: Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1−((E)/(C)) times 100.

The compounds of the invention that were tested in the above assay exhibited IC₅₀ values of less than 10 μM.

Dopamine Turnover: Rats were injected s.c. or p.o. (gavage) and then decapitated either 1 or 2 hours later. Nucleus accumbens was rapidly dissected (2 mm slices, 4° C., in 0.32 M sucrose), placed in 0.1 N perchloric acid, and then homogenized. After centrifugation 10 uL of the supernatant was assayed by HPLC-ECD. Turnover/utilization of dopamine (DA) was calculated as the ratio of tissue concentrations of metabolites ([DOPAC]+[HVA]) to DA and expressed as percent of control.

### Pharmacological Testing of CB-1 Receptor Antagonists

The utility of the compounds of the present invention in the practice of the instant invention can be evidenced by activity in at least one of the protocols described hereinbelow. The following acronyms are used in the protocols described below.

BSA—bovine serum albumin

DMSO—dimethylsulfoxide

EDTA—ethylenediamine tetracetic acid

PBS—phosphate-buffered saline

EGTA—ethylene glycol-bis(β-aminoethyl ether) N,N,N′,N′-tetraacetic acid

GDP—guanosine diphosphate

sc—subcutaneous

po—orally

ip—intraperitoneal

icv—intra cerebro ventricular

iv—intravenous

[³H]SR141716A—radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.

[³H]CP-55940—radiolabled 5-(1,1-dimethylheptyl)-2-[5-hydroxy-2-(3-hydroxypropyl)-cyclohexyl]-phenol available from NEN Life Science Products, Boston, Mass.

AM251—N-(piperidin-1-yl)-1-(2,4-dichlorophenyl)-5-(4-iodophenyl)-4-methyl-1H-pyrazole-3-carboxamide available from Tocris™, Ellisville, Mo.

In Vitro Biological Assays

Bioassay systems for determining the CB-1 and CB-2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

The following assays were designed to detect compounds that inhibit the binding of [³H] SR141716A (selective radiolabeled CB-1 ligand) and [³H] 5-(1,1-dimethylheptyl)-2-[5-hydroxy-2-(3-hydroxypropyl)-cyclohexyl]-phenol ([³H]CP-55940; radiolabeled CB-1/CB-2 ligand) to their respective receptors.

Rat CB-1 Receptor Binding Protocol

PelFreeze brains (available from Pel Freeze Biologicals, Rogers, Ark.) were cut up and placed in tissue preparation buffer (5 mM Tris HCl, pH=7.4 and 2 mM EDTA), polytroned at high speed and kept on ice for 15 minutes. The homogenate was then spun at 1,000×g for 5 minutes at 4° C. The supernatant was recovered and centrifuged at 100,000×G for 1 hour at 4° C. The pellet was then re-suspended in 25 ml of TME (25 nM Tris, pH=7.4, 5 mM MgCl₂, and 1 mM EDTA) per brain used. A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [³H] SR141716A was diluted in a ligand buffer (0.5% BSA plus TME) and 25 μl were added to the plate. A BCA protein assay was used to determine the appropriate tissue concentration and then 200 μl of rat brain tissue at the appropriate concentration was added to the plate. The plates were covered and placed in an incubator at 20° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

Human CB-1 Receptor Binding Protocol

Human embryonic kidney 293 (HEK 293) cells transfected with the CB-1 receptor cDNA (obtained from Dr. Debra Kendall, University of Connecticut) were harvested in homogenization buffer (10 mM EDTA, 10 mM EGTA, 10 mM Na Bicarbonate, protease inhibitors; pH=7.4), and homogenized with a Dounce Homogenizer. The homogenate was then spun at 1,000×g for 5 minutes at 4° C. The supernatant was recovered and centrifuged at 25,000×G for 20 minutes at 4° C. The pellet was then re-suspended in 10 ml of homogenization buffer and re-spun at 25,000×G for 20 minutes at 4° C. The final pellet was re-suspended in 1 ml of TME (25 mM Tris buffer (pH=7.4) containing 5 mM MgCl₂ and 1 mM EDTA). A protein assay was performed and 200 μl of tissue totaling 20 lag was added to the assay.

The test compounds were diluted in drug buffer (0.5% BSA, 10% DMSO and TME) and then 25 μl were added to a deep well polypropylene plate. [3H]SR141716A was diluted in a ligand buffer (0.5% BSA plus TME) and 25 μl were added to the plate. The plates were covered and placed in an incubator at 30° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

CB-2 Receptor Binding Protocol

Chinese hamster ovary-K1 (CHO-K1) cells transfected with CB-2 cDNA (obtained from Dr. Debra Kendall, University of Connecticut) were harvested in tissue preparation buffer (5 mM Tris-HCl buffer (pH=7.4) containing 2 mM EDTA), polytroned at high speed and kept on ice for 15 minutes. The homogenate was then spun at 1,000×g for 5 minutes at 4° C. The supernatant was recovered and centrifuged at 100,000×G for 1 hour at 4° C. The pellet was then re-suspended in 25 ml of TME (25 mM Tris buffer (pH=7.4) containing 5 mM MgCl₂ and 1 mM EDTA) per brain used. A protein assay was performed and 200 μl of tissue totaling 10 μg was added to the assay.

The test compounds were diluted in drug buffer (0.5% BSA, 10% DMSO, and 80.5% TME) and then 25 μl were added to the deep well polypropylene plate. [3H] CP-55940 was diluted a ligand buffer (0.5% BSA and 99.5% TME) and then 25 μl were added to each well at a concentration of 1 nM. A BCA protein assay was used to determine the appropriate tissue concentration and 200 μl of the tissue at the appropriate concentration was added to the plate. The plates were covered and placed in an incubator at 30° C. for 60 minutes. At the end of the incubation period 250 μl of stop buffer (5% BSA plus TME) was added to the reaction plate. The plates were then harvested by Skatron format onto GF/B filtermats presoaked in BSA (5 mg/ml) plus TME. Each filter was washed twice. The filters were dried overnight. The filters were then counted on the Wallac Betaplate™ counter.

CB-1 GTPγ [³⁵S] Binding Assay

Membranes were prepared from CHO-K1 cells stably transfected with the human CB-1 receptor cDNA. Membranes were prepared from cells as described by Bass et al, in “Identification and characterization of novel somatostatin antagonists,” Molecular Pharmacology, 50, 709-715 (1996). GTPγ [³⁵S] binding assays were performed in a 96 well FlashPlate™ format in duplicate using 100 μM GTPγ[³⁵S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix was then incubated with increasing concentrations of antagonist (10⁻¹⁰ M to 10⁻⁵ M) for 10 minutes and challenged with the cannabinoid agonist CP-55940 (10 μM). Assays were performed at 30° C. for one hour. The FlashPlates™ were then centrifuged at 2000×g for 10 minutes. Stimulation of GTPγ[³⁵S] binding was then quantified using a Wallac Microbeta EC₅₀ calculations done using Prism™ by Graphpad.

Inverse agonism was measured in the absense of agonist.

CB-1 FLIPR-Based Functional Assay Protocol

CHO-K1 cells co-transfected with the human CB-1 receptor cDNA (obtained from Dr. Debra Kendall, University of Connecticut) and the promiscuous G-protein G16 were used for this assay. Cells were plated 48 hours in advance at 12500 cells per well on collagen coated 384 well black clear assay plates. Cells were incubated for one hour with 4 μM Fluo-4 AM (Molecular Probes) in DMEM (Gibco) containing 2.5 mM probenicid and pluronic acid (0.04%). The plates were then washed 3 times with HEPES-buffered saline (containing probenicid; 2.5 mM) to remove excess dye. After 20 min the plates were added to the FLIPR individually and fluorescence levels was continuously monitored over an 80 s period. Compound additions were made simultaneously to all 384 wells after 20 s of baseline. Assays were performed in triplicate and 6 point concentration-response curves generated. Antagonist compounds were subsequently challenged with 3 μM WIN 55,212-2 (agonist). Data were analyzed using Graph Pad Prism.

Detection of Inverse Agonists

The following cyclic-AMP assay protocol using intact cells was used to determine inverse agonist activity.

Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C.

Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

In Vivo Biological Assays

Cannabinoid agoinists such as Δ⁹-tetrahydrocannabinol (Δ⁹-THC) and CP-55940 have been shown to affect four characteristic behaviors in mice, collectively known as the Tetrad. For a description of these behaviors see: Smith, P. B., et al. in “The pharmacological activity of anandamide, a putative endogenous cannabinoid, in mice.” J. Pharmacol. Exp. Ther., 270(1), 219-227 (1994) and Wiley, J., et al. in “Discriminative stimulus effects of anandamide in rats,” Eur. J. Pharmacol., 276(1-2), 49-54 (1995). Reversal of these activities in the Locomotor Activity, Catalepsy, Hypothermia, and Hot Plate assays described below provides a screen for in vivo activity of CB-1 antagonists.

All data is presented as % reversal from agonist alone using the following formula: (CP/agonist−vehicle/agonist)/(vehicle/vehicle−vehicle/agonist). Negative numbers indicate a potentiation of the agonist activity or non-antagonist activity. Positive numbers indicate a reversal of activity for that particular test.

Locomotor Activity

Male ICR mice (n=6) (17-19 g, Charles River Laboratories, Inc., Wilmington, Mass.) were pre-treated with test compound (sc, po, ip, or icv). Fifteen minutes later, the mice were challenged with CP-55940 (sc). Twenty-five minutes after the agonist injection, the mice were placed in clear acrylic cages (431.8 cm×20.9 cm×20.3 cm) containing clean wood shavings. The subjects were allowed to explore surroundings for a total of about 5 minutes and the activity was recorded by infrared motion detectors (available from Coulbourn Instruments™, Allentown, Pa.) that were placed on top of the cages. The data was computer collected and expressed as “movement units.”

Catalepsy

Male ICR mice (n=6)(17-19 g upon arrival) were pre-treated with test compound (sc, po, ip or icv). Fifteen minutes later, the mice were challenged with CP-55940 (sc). Ninety minutes post injection, the mice were placed on a 6.5 cm steel ring attached to a ring stand at a height of about 12 inches. The ring was mounted in a horizontal orientation and the mouse was suspended in the gap of the ring with fore- and hind-paws gripping the perimeter. The duration that the mouse remained completely motionless (except for respiratory movements) was recorded over a 3-minute period.

The data were presented as a percent immobility rating. The rating was calculated by dividing the number of seconds the mouse remains motionless by the total time of the observation period and multiplying the result by 100. A percent reversal from the agonist was then calculated.

Hypothermia

Male ICR mice (n=5) (17-19 g upon arrival) were pretreated with test compounds (sc, po, ip or icv). Fifteen minutes later, mice were challenged with the cannabinoid agonist CP-55940 (sc). Sixty-five minutes post agonist injection, rectal body temperatures were taken. This was done by inserting a small thermostat probe approximately 2-2.5 cm into the rectum. Temperatures were recorded to the nearest tenth of a degree

Hot Plate

Male ICR mice (n=7) (17-19 g upon arrival) are pre-treated with test compounds (sc, po, ip or iv). Fifteen minutes later, mice were challenged with a cannabinoid agonist CP-55940 (sc). Forty-five minutes later, each mouse was tested for reversal of analgesia using a standard hot plate meter (Columbus Instruments). The hot plate was 10″×10″×0.75″ with a surrounding clear acrylic wall. Latency to kick, lick or flick hindpaw or jump from the platform was recorded to the nearest tenth of a second. The timer was experimenter activated and each test had a 40 second cut off. Data were presented as a percent reversal of the agonist induced analgesia.

Food Intake

The following screen was used to evaluate the efficacy of test compounds for inhibiting food intake in Sprague-Dawley rats after an overnight fast.

Male Sprague-Dawley rats were obtained from Charles River Laboratories, Inc. (Wilmington, Mass.). The rats were individually housed and fed powdered chow. They were maintained on a 12 hour light/dark cycle and received food and water ad libitum. The animals were acclimated to the vivarium for a period of one week before testing was conducted. Testing was completed during the light portion of the cycle.

To conduct the food intake efficacy screen, rats were transferred to individual test cages without food the afternoon prior to testing, and the rats were fasted overnight. After the overnight fast, rats were dosed the following morning with vehicle or test compounds. A known antagonist was dosed (3 mg/kg) as a positive control, and a control group received vehicle alone (no compound). The test compounds were dosed at ranges between 0.1 and 100 mg/kg depending upon the compound. The standard vehicle was 0.5% (w/v) methylcellulose in water and the standard route of administration was oral. However, different vehicles and routes of administration were used to accommodate various compounds when required. Food was provided to the rats 30 minutes after dosing and the Oxymax automated food intake system (Columbus Instruments, Columbus, Ohio) was started. Individual rat food intake was recorded continuously at 10-minute intervals for a period of two hours. When required, food intake was recorded manually using an electronic scale; food was weighed every 30 minutes after food was provided up to four hours after food was provided. Compound efficacy was determined by comparing the food intake pattern of compound-treated rats to vehicle and the standard positive control.

Alcohol Intake

The following protocol evaluates the effects of alcohol intake in alcohol preferring (P) female rats (bred at Indiana University) with an extensive drinking history. The following references provide detailed descriptions of P rats: Li, T.-K., et al., “Indiana selection studies on alcohol related behaviors” in Development of Animal Models as Pharmacogenetic Tools (eds McClearn C. E., Deitrich R. A. and Erwin V. G.), Research Monograph 6, 171-192 (1981) NIAAA, ADAMHA, Rockville, Md.; Lumeng, L, et al., “New strains of rats with alcohol preference and nonpreference” Alcohol And Aldehyde Metabolizing Systems, 3, Academic Press, New York, 537-544 (1977); and Lumeng, L, et al., “Different sensitivities to ethanol in alcohol-preferring and -nonpreferring rats,” Pharmacol, Biochem Behav., 16, 125-130 (1982).

Female rats were given 2 hours of access to alcohol (10% v/v and water, 2-bottle choice) daily at the onset of the dark cycle. The rats were maintained on a reverse cycle to facilitate experimenter interactions. The animals were initially assigned to four groups equated for alcohol intakes: Group 1—vehicle (n=8); Group 2—positive control (e.g. 5.6 mg/kg AM251; n=8); Group 3—low dose test compound (n=8); and Group 4—high dose of test compound (n=8). Test compounds were generally mixed into a vehicle of 30% (w/v) β-cyclodextrin in distilled water at a volume of 1-2 ml/kg. Vehicle injections were given to all groups for the first two days of the experiment. This was followed by 2 days of drug injections (to the appropriate groups) and a final day of vehicle injections. On the drug injection days, drugs were given sc 30 minutes prior to a 2-hour alcohol access period. Alcohol intake for all animals was measured during the test period and a comparison was made between drug and vehicle-treated animals to determine effects of the compounds on alcohol drinking behavior.

Additional drinking studies were done utilizing female C57BI/6 mice (Charles River). Several studies have shown that this strain of mice will readily consume alcohol with little to no manipulation required (Middaugh et al., “Ethanol Consumption by C57BU6 Mice: Influence of Gender and Procedural Variables” Alcohol, 17 (3), 175-183, 1999; Le et al., “Alcohol Consumption by C57BU6, BALA/c, and DBA/2 Mice in a Limited Access Paradigm” Pharmacology Biochemisrty and Behavior, 47, 375-378, 1994).

For our purposes, upon arrival (17-19 g) mice were individually housed and given unlimited access to powdered rat chow, water and a 10% (w/v) alcohol solution. After 2-3 weeks of unlimited access, water was restricted for 20 hours and alcohol was restricted to only 2 hours access daily. This was done in a manner that the access period was the last 2 hours of the dark part of the light cycle.

Once drinking behavior stabilized, testing commenced. Mice were considered stable when the average alcohol consumption for 3 days was ±20% of the average for all 3 days. Day 1 of test consisted of all mice receiving vehicle injection (sc or ip). Thirty to 120 minutes post injection access was given to alcohol and water. Alcohol consumption for that day was calculated (g/kg) and groups were assigned (n=7-10) so that all groups had equivocal alcohol intake. On day 2 and 3, mice were injected with vehicle or test compound and the same protocol as the previous day was followed. Day 4 was wash-out and no injections were given. Data was analyzed using repeated measures ANOVA. Change in water or alcohol consumption was compared back to vehicle for each day of the test. Positive results would be interpreted as a compound that was able to significantly reduce alcohol consumption while having no effect on water

Oxygen Consumption

Methods:

Whole body oxygen consumption is measured using an indirect calorimeter (Oxymax from Columbus Instruments, Columbus, Ohio) in male Sprague Dawley rats (if another rat strain or female rats are used, it will be specified). Rats (300-380 g body weight) are placed in the calorimeter chambers and the chambers are placed in activity monitors. These studies are done during the light cycle. Prior to the measurement of oxygen consumption, the rats are fed standard chow ad libitum. During the measurement of oxygen consumption, food is not available. Basal pre-dose oxygen consumption and ambulatory activity are measured every 10 minutes for 2.5 to 3 hours. At the end of the basal pre-dosing period, the chambers are opened and the animals are administered a single dose of compound (the usual dose range is 0.001 to 10 mg/kg) by oral gavage (or other route of administration as specified, i.e. s.c., i.p., i.v.). Drugs are prepared in methylcellulose, water or other specified vehicle (examples include PEG400, 30% beta-cyclodextran and propylene glycol). Oxygen consumption and ambulatory activity are measured every 10 minutes for an additional 1-6 hours post-dosing.

The Oxymax calorimeter software calculates the oxygen consumption (ml/kg/h) based on the flow rate of air through the chambers and difference in oxygen content at inlet and output ports. The activity monitors have 15 infrared light beams spaced one inch apart on each axis, ambulatory activity is recorded when two consecutive beams are broken and the results are recorded as counts.

Resting oxygen consumption, during pre- and post-dosing, is calculated by averaging the 10-min O₂ consumption values, excluding periods of high ambulatory activity (ambulatory activity count >100) and excluding the first 5 values of the pre-dose period and the first value from the post-dose period. Change in oxygen consumption is reported as percent and is calculated by dividing the post-dosing resting oxygen consumption by the pre-dose oxygen consumption*100. Experiments will typically be done with n=4-6 rats and results reported are mean+/−SEM.

Interpretation:

An increase in oxygen consumption of >10% is considered a positive result. Historically, vehicle-treated rats have no change in oxygen consumption from pre-dose basal.

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising a NRPA as described above and a CB-1 receptor antagonist as described above in a pharmaceutically acceptable carrier can be administered.

The amount and timing of compounds administered will, of course, be based on the judgement of the prescribing physician. Thus, because of patient to patient variability, the dosages given below are a guideline and the physician may titrate doses of the agent to achieve the activity that the physician considers appropriate for the individual patient. In considering the degree of activity desired, the physician must balance a variety of factors such as cognitive function, age of the patient, presence of preexisting disease, as well as presence of other diseases (e.g., cardiovascular). The following paragraphs provide preferred dosage ranges for the various components of this invention (based on average human weight of 70 kg).

In general, an effective dosage for the NRPA in the range of 0.001 to 200 mg/kg/day, preferably 0.005 to 10.0 mg/kg/day.

In general, an effective dosage for the CB-1 receptor antagonists when used in the combination compositions and methods of this invention, is in the range of 0.001 to 200 mg/kg/day, preferably 0.005 to 10.0 mg/kg/day.

The compositions of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral or transdermal dosage form.

For oral administration a pharmaceutical composition can take the form of solutions, suspensions, tablets, pills, capsules, powders, and the like. Tablets containing various excipient such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the dependence of the subject being treated.

1. A pharmaceutical composition for treating nicotine dependence or addiction, tobacco dependence or addiction, reducing nicotine withdrawal symptoms or aiding in the cessation or lessening of tobacco use or substance abuse or behavioral dependencies, comprising:
(a) a nicotinic receptor partial agonist or a pharmaceutically acceptable salt thereof;
(b) a CB-1 receptor antagonist or pharmaceutically acceptable salt thereof; and
(c) a pharmaceutically acceptable carrier;
wherein the active agents “a” and “b” above are present in amounts that render the composition effective in treating nicotine dependence or addiction, tobacco dependence or addiction, reducing nicotine withdrawal symptoms or aiding in the cessation or lessening of tobacco use or substance abuse or behavioral dependencies.

2. The pharmaceutical composition according to claim 1, wherein said CB-1 receptor antagonist is selected from 1-[9-(4-chloro-phenyl)-8-(2-chlorophenyl)-9H-purin-6-yl]-3-ethylamino-azetidine-3-carboxylic acid amide; 1-[9-(4-chloro-phenyl)-8-(2-chlorophenyl)-9H-purin-6-yl]-3-ethylamino-azetidine-3-carboxylic acid amide; 1-[9-(4-chlorophenyl)-8-(2-chlorophenyl)-9H-purin-6-yl]-3-isopropylaminoazetidine-3-carboxylic acid amide; 1-{1-[9-(4-chlorophenyl)-8-(2-chlorophenyl)-9H-purin-6-yl]-4-phenylpiperidin-4-yl}-ethanone; {3-[9-(4-chlorophenyl)-8-(2,4-dichlorophenyl)-9H-purin-6-yl]-3-(1α,5α,6α)-azabicyclo[3.1.0]hex-6-yl}-dimethylamine; 6-(1-benzylpyrrolidin-3-yloxy)-9-(4-chlorophenyl)-8-(2,4-dichlorophenyl)-9H-purine; 9-(4-chlorophenyl)-6-(1-cyclohexylazetidin-3-yloxy)-8-(2,4-dichlorophenyl)-9H-purine; 6-tert-butoxy-9-(4-chlorophenyl)-8-(2,4-dichlorophenyl)-9H-purine; 9-(4-chlorophenyl)-8-(2,4-dichlorophenyl)-6-isopropoxy-9H-purine; 1-[9-(4-chlorophenyl)-8-(2,4-dichlorophenyl)-9H-purin-6-yl]-4-propylaminopiperidine-4-carboxylic acid amide; 1-[9-(4-chlorophenyl)-8-(2-fluorophenyl)-9H-purin-6-yl]-4-propylaminopiperidine-4-carboxylic acid amide; 1-[9-(4-chlorophenyl)-8-(2-chlorophenyl)-9H-purin-6-yl]-4-propylaminopiperidine-4-carboxylic acid amide; 1-[9-(4-chlorophenyl)-8-(2-fluorophenyl)-2-methyl-9H-purin-6-yl]-4-isopropylaminopiperidine-4-carboxylic acid amide; 1-[9-(4-chlorophenyl)-8-(2-chlorophenyl)-9H-purin-6-yl]-4-pyrrolidin-1-yl-piperidine-4-carboxylic acid amide; 1-[9-(4-chlorophenyl)-8-(2-chlorophenyl)-9H-purin-6-yl]-4-ethylamino-piperidine-4-carboxylic acid amide; 1-[9-(4-chlorophenyl)-8-(2-chlorophenyl)-9H-purin-6-yl]-4-isopropylaminopiperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 9-[9-(4-chlorophenyl)-8-(2-chlorophenyl)-9H-purin-6-yl]-1-methyl-4-oxa-1,9-diazaspiro[5.5]undecan-2-one; 8-[9-(4-chlorophenyl)-8-(2,4-dichlorophenyl)-9H-purin-6-yl]-1-isopropyl-1,3,8-triazaspiro[4.5]decan-4-one; 1-[9-(4-chlorophenyl)-8-(2-chlorophenyl)-9H-purin-6-yl]-4-(4-fluorophenyl)-piperidin-4-ol; 1-[9-(4-chlorophenyl)-8-(2-chlorophenyl)-9H-purin-6-yl]-4-phenylpiperidin-4-ol; 4-benzyl-1-[9-(4-chlorophenyl)-8-(2-chlorophenyl)-9H-purin-6-yl]-piperidin-4-ol; 4-[9-(4-chlorophenyl)-8-(2-chlorophenyl)-9H-purin-6-yl]-piperazine-2-carboxylic acid methylamide; 9-(4-chlorophenyl)-8-(2,4-dichlorophenyl)-6-(4-pyridin-2-yl-piperazin-1-yl)-9H-purine; and 9-(4-chlorophenyl)-8-(2,4-dichlorophenyl)-6-(4-pyrimidin-2-yl-piperazin-1-yl)-9H-purine; 1-[9-(4-chlorophenyl)-8-(2-fluorophenyl)-9H-purin-6-yl]-4-isopropylamino-piperidine-4-carboxylic acid amide; 1-[9-(4-chlorophenyl)-8-(2-chlorophenyl)-9H-purin-6-yl]-4-isopropylamino-piperidine-4-carboxylic acid amide; 4-amino-1-[9-(4-chlorophenyl)-8-(2-chlorophenyl)-9H-purin-6-yl]-piperidine-4-carboxylic acid amide; 1-[9-(4-chlorophenyl)-8-(2-chlorophenyl)-9H-purin-6-yl]-4-ethylamino-piperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 4-amino-1-[9-(4-chloro-phenyl)-8-(2-chlorophenyl)-9H-purin-6-yl]-piperidine-4-carboxylic acid amide; and 1-[9-(4-chlorophenyl)-8-(2-chlorophenyl)-9H-purin-6-yl]4-ethylaminopiperidine-4-carboxylic acid amide; and a pharmaceutically acceptable salt thereof or a solvate or hydrate of said compound or said salt.

3. The pharmaceutical composition according to claim 1, wherein the CB-1 receptor antagonist is selected from 7-(2-chlorophenyl)-8-(4-chlorophenyl)-2-methyl-4-(4-methylpiperazin-1-yl)-pyrazolo[1,5-a][1,3,5]triazine; 7-(2-chlorophenyl)-8-(4-chlorophenyl)-2-methyl-4-(4-pyrimidin-2-ylpiperazin-1-yl)-pyrazolo[1,5-a][1,3,5]triazine; 7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and 7-(2-chlorophenyl)-8-(4-chlorophenyl)-2-methyl-4-[4-(propane-2-sulfonyl)-piperazin-1-yl]-pyrazolo[1,5-a][1,3,5]triazine; 1-[7-(2-chlorophenyl)-8-(4-chlorophenyl)-2-methylpyrazolo[1,5-a][1,3,5]triazin-4-yl]-4-methylaminopiperidine-4-carboxylic acid amide; 1-[7-(2-chlorophenyl)-8-(4-fluorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-isopropylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; 1-[7-(2-chlorophenyl)-8-(4-chlorophenyl)-2-methylpyrazolo[1,5-a][1,3,5]triazin-4-yl]-3-methylaminoazetidine-3-carboxylic acid amide; and 1-[7-(2-chlorophenyl)-8-(4-chlorophenyl)-2-methylpyrazolo[1,5-a][1,3,5]triazin-4-yl]-3-dimethylaminoazetidine-3-carboxylic acid amide; 1-{1-[7-(2-chlorophenyl)-8-(4-chlorophenyl)-2-methylpyrazolo[1,5-a][1,3,5]triazin-4-yl]-4-phenylpiperidin-4-yl}-ethanone; 3-[7-(2-chlorophenyl)-8-(4-chlorophenyl)-2-methylpyrazolo[1,5-a][1,3,5]triazin-4-yl]-3-azabicyclo[3.1.0]hex-6-ylamine; 1-[7-(2-chlorophenyl)-8-(4-chlorophenyl)-2-methylpyrazolo[1,5-a][1,3,5]triazin-4-yl]-4-(4-fluorophenyl)-piperidin-4-ol; 4-benzyl-1-[7-(2-chlorophenyl)-8-(4-chlorophenyl)-2-methylpyrazolo[1,5-a][1,3,5]triazin-4-yl]-piperidin-4-ol; 2-[7-(2-chlorophenyl)-8-(4-chlorophenyl)-2-methylpyrazolo[1,5-a][1,3,5]triazin-4-yl]-5-methyl-2,5,7-triazaspiro[3.4]octan-8-one; 2-[7-(2-chlorophenyl)-8-(4-chlorophenyl)-2-methylpyrazolo[1,5-a][1,3,5]triazin-4-yl]-2,5,7-triazaspiro[3.4]octan-8-one; 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; 2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one; 4-(1-benzylpyrrolidin-3-yloxy)-7-(2-chlorophenyl)-8-(4-chlorophenyl)-2-methylpyrazolo[1,5-a][1,3,5]triazine; 7-(2-chlorophenyl)-8-(4-chlorophenyl)-4-(1-cyclohexylazetidin-3-yloxy)-2-methylpyrazolo[1,5-a][1,3,5]triazine; 7-(2-chlorophenyl)-8-(4-chlorophenyl)-4-isopropoxy-2-methylpyrazolo[1,5-a][1,3,5]triazine; and 4-tert-butoxy-7-(2-chlorophenyl)-8-(4-chlorophenyl)-2-methylpyrazolo[1,5-a][1,3,5]triazine; butyl-[7-(2-chlorophenyl)-8-(4-chlorophenyl)-2-methylpyrazolo[1,5-a][1,3,5]triazin-4-yl]-amine; 7-(2-chlorophenyl)-8-(4-chlorophenyl)-2-methyl-4-piperidin-1-yl-pyrazolo[1,5-a][1,3,5]triazine; [7-(2-chlorophenyl)-8-(4-chlorophenyl)-2-methylpyrazolo[1,5-a][1,3,5]triazin-4-yl]-[2-(4-fluorophenyl)-ethyl]-amine; 7-(2-chlorophenyl)-8-(4-chlorophenyl)-2-methyl-4-morpholin-4-yl-pyrazolo[1,5-a][1,3,5]triazine; and [7-(2-chlorophenyl)-8-(4-chlorophenyl)-2-methylpyrazolo[1,5-a][1,3,5]triazin-4-yl]-(2-morpholin-4-yl-ethyl)-amine; 1-[7-(2-chlorophenyl)-8-(4-chlorophenyl)-2-methylpyrazolo[1,5-a][1,3,5]triazin-4-yl]4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; and 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and a pharmaceutically acceptable salt thereof or a solvate or hydrate of said compound or said salt.

4. The pharmaceutical composition according to claim 1, wherein said CB-1 receptor antagonist is selected from 3-(4-chlorophenyl)-2-(2-chlorophenyl)-7-(4-methyl-piperazin-1-yl)-pyrazolo[1,5-a]pyrimidine; 3-(4-chlorophenyl)-2-(2-chlorophenyl)-7-(4-pyrimidin-2-yl-piperazin-1-yl)-pyrazolo[1,5-a]pyrimidine; 3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and 3-(4-chlorophenyl)-2-(2-chlorophenyl)-7-[4-(propane-2-sulfonyl)-piperazin-1-yl]-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; 1-[3-(4-chlorophenyl)-2-(2-chlorophenyl)-pyrazolo[1,5-a]pyrimidin-7-yl]-3-ethylaminoazetidine-3-carboxylic acid amide; 3-amino-1-[3-(4-chlorophenyl)-2-(2-chlorophenyl)-pyrazolo[1,5-a]pyrimidin-7-yl]-azetidine-3-carboxylic acid amide; 1-[3-(4-chlorophenyl)-2-(2-chlorophenyl)-6-methylpyrazolo[1,5-a]pyrimidin-7-yl]-3-ethylaminoazetidine-3-carboxylic acid amide; 1-[3-(4-chlorophenyl)-2-(2-chlorophenyl)-pyrazolo[1,5-a]pyrimidin-7-yl]-3-isopropylaminoazetidine-3-carboxylic acid amide; 1-[3-(4-chlorophenyl)-2-(2-chlorophenyl)-5,6-dimethylpyrazolo[1,5-a]pyrimidin-7-yl]-3-ethylamino-azetidine-3-carboxylic acid amide; 1-[3-(4-chlorophenyl)-2-(2-chlorophenyl)-pyrazolo[1,5-a]pyrimidin-7-yl]-3-methylaminoazetidine-3-carboxylic acid amide; 1-[3-(4-chlorophenyl)-2-(2-chlorophenyl)-5-methylpyrazolo[1,5-a]pyrimidin-7-yl]-3-ethylaminoazetidine-3-carboxylic acid amide; 1-{1-[3-(4-chlorophenyl)-2-(2-chlorophenyl)-pyrazolo[1,5-a]pyrimidin-7-yl]-4-phenylpiperidin-4-yl}-ethanone; 3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine; 1-[3-(4-chlorophenyl)-2-(2-chlorophenyl)-pyrazolo[1,5-a]pyrimidin-7-yl]-4-(4-fluorophenyl)-piperidin-4-ol; 4-benzyl-1-[3-(4-chlorophenyl)-2-(2-chlorophenyl)-pyrazolo[1,5-a]pyrimidin-7-yl]-piperidin-4-ol; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-2,5,7-triazaspiro[3.4]octan-8-one; 8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-5-methyl-2,5,7-triazaspiro[3.4]octan-8-one; 7-(1-benzylpyrrolidin-3-yloxy)-3-(4-chlorophenyl)-2-(2-chlorophenyl)-pyrazolo[1,5-a]pyrimidine; and 3-(4-chlorophenyl)-2-(2-chlorophenyl)-7-(1-cyclohexylazetidin-3-yloxy)-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; and 1-[3-(4-chlorophenyl)-2-(2-chlorophenyl)-pyrazolo[1,5-a]pyrimidin-7-yl]-3-ethylaminoazetidine-3-carboxylic acid amide; and a pharmaceutically acceptable salt thereof or a solvate or hydrate of said compound or said salt.

5. The pharmaceutical composition according to claim 1, wherein said CB-1 receptor antagonist is selected from 5-(4-chloro-phenyl)-3-(5-cyclohexyl-1H-imidazol-2-yl)-1-(2,4-dichloro-phenyl)-4-methyl-1H-pyrazole; 5-(4-chloro-phenyl)-3-(2-cyclohexyl-3H-imidazol-4-yl)-1-(2,4-dichloro-phenyl)-4-methyl-1H-pyrazole; 5-(4-chloro-phenyl)-1-(2,4-dichloro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-4-methyl-3-[1-(1-phenyl-ethyl)-1H-imidazol-4-yl]-1H-pyrazole; 5-(4-chloro-phenyl)-1-(2-fluoro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole: 5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and 1-(2-chloro-phenyl)-5-(4-chloro-phenyl)-4-methyl-3-[1-(1-methyl-1-phenyl-ethyl)-1H-imidazol-4-yl]-1H-pyrazole; and a pharmaceutically acceptable salt thereof or a solvate or hydrate of the compound or the salt.

6. The pharmaceutical composition according to claim 1, wherein said CB-1 receptor antagonist is selected from 1-[5-(4-chloro-phenyl)-1-(2-chloro-phenyl)-4-methyl-1H-pyrazol-3-yl]-2-piperidin-1-yl-ethanone; 1-[5-(4-chloro-phenyl)-1-(2-chloro-phenyl)-4-methyl-1H-pyrazol-3-yl]-2-morpholin-4-yl-ethanone; 1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone; 1-[5-(4-chloro-phenyl)-1-(2-chloro-phenyl)-4-methyl-1H-pyrazol-3-yl]-2-[4-(1-methyl-cyclopropanecarbonyl)-piperazin-1-yl]-ethanone; N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide; 1-[5-(4-chloro-phenyl)-1-(2-fluoro-phenyl)-4-methyl-1H-pyrazol-3-yl]-2-morpholin-4-yl-ethanone; 1-[5-(4-chloro-phenyl)-1-(2-fluoro-phenyl)-4-methyl-1H-pyrazol-3-yl]-2-piperidin-1-yl-ethanone; 1-[5-(4-chloro-phenyl)-1-(2-fluoro-phenyl)-4-methyl-1H-pyrazol-3-yl]-2-(4-trifluoroacetyl-piperazin-1-yl)-ethanone; 1-[1-(2-chloro-phenyl)-5-(4-chloro-phenyl)-4-methyl-1H-pyrazol-3-yl]-2-pyrrolidin-1-yl-ethanone; 1-[1-(2-chloro-phenyl)-5-(4-chloro-phenyl)-4-methyl-1H-pyrazol-3-yl]-2-[1,4]oxazepan-4-yl-ethanone; and 1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and a pharmaceutically acceptable salt thereof, a prodrug of the compound or the salt, or a solvate or hydrate of the compound.

7. The pharmaceutical composition according claim 1, wherein said CB-1 receptor antagonist is selected from 2-(benzyl-isopropyl-amino)-1-[1-(2-chloro-phenyl)-5-(4-chloro-phenyl)-4-methyl-1H-pyrazol-3-yl]-ethanol; 1-[5-(4-chloro-phenyl)-1-(2-chloro-phenyl)-4-methyl-1H-pyrazol-3-yl]-2-(3,5-dimethyl-piperidin-1-yl)-ethanol; 1-{2-[1-(2-chloro-phenyl)-5-(4-chloro-phenyl)-4-methyl-1H-pyrazol-3-yl]-2-hydroxy-ethyl}-4-isopropylamino-piperidine-4-carboxylic acid amide; 1-[5-(4-chloro-phenyl)-1-(2-chloro-phenyl)-4-methyl-1H-pyrazol-3-yl]-2-(3,3-dimethyl-piperidin-1-yl)-ethanol; 1-[5-(4-chloro-phenyl)-1-(2-chloro-phenyl)-4-methyl-1H-pyrazol-3-yl]-2-piperidin-1-yl-ethanol; and 1-[5-(4-chloro-phenyl)-1-(2-chloro-phenyl)-4-methyl-1H-pyrazol-3-yl]-2-morpholin-4-yl-ethanol; and a pharmaceutically acceptable salt thereof, a prodrug of the compound or the salt, or a solvate or hydrate of the compound.

8. The pharmaceutical composition according to claim 1 wherein said CB-1 receptor antagonist is selected from 2-[5-(4-chloro-phenyl)-1-(2-chloro-phenyl)-4-methyl-1H-pyrazol-3-yl]-4-cyclohexyl-morpholine; 2-[5-(4-chloro-phenyl)-1-(2-chloro-phenyl)-4-methyl-1H-pyrazol-3-yl]-4-(propane-2-sulfonyl)-morpholine; 2-[5-(4-chloro-phenyl)-1-(2-chloro-phenyl)-4-methyl-1H-pyrazol-3-yl]-4-(toluene-4-sulfonyl)-morpholine; 1-{2-[1-(2-chloro-phenyl)-5-(4-chloro-phenyl)-4-methyl-1H-pyrazol-3-yl]-morpholin-4-yl}-2-methyl-propan-1-one; and 2-[1-(2-chloro-phenyl)-5-(4-chloro-phenyl)-4-methyl-1H-pyrazol-3-yl]-4-(4-trifluoromethyl-benzyl)-morpholine; and a pharmaceutically acceptable salt thereof or a solvate or hydrate of the compound.

9. The pharmaceutical composition according claim 1, wherein said CB-1 receptor antagonist is selected from 1-[1-(4-chloro-phenyl)-2-(2,4-dichloro-phenyl)-5-methyl-1H-imidazol-4-yl]-2-piperidin-1-yl-ethanone and 1-[1-(4-chloro-phenyl)-2-(2,4-dichloro-phenyl)-5-methyl-1H-imidazol-4-yl]-2-morpholin-4-yl-ethanone; and a pharmaceutically acceptable salt thereof, or a solvate or hydrate of the compound.

10. The pharmaceutically composition according to claim 1, wherein said nicotinic receptor partial agonist is selected from:
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
9-vinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2-a][1,5]diazocin-8-one;
3-benzyl-9-bromo-1,2,3,4,5,6-hexahydro-1,5-methano-pyrido[1,2-a][1,5]diazocin-8-one;
3-benzyl-9-chloro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-ethynyl-1,2,3,4,5,6-hexahydro-1,5-methano-pyrido[1,2a][1,5]diazocin-8-one;
9-(2-propenyl)-1,2,3,4,5,6-hexahydro-1,5-methano-pyrido[1,2a][1,5]diazocin-8-one;
9-(2-propyl)-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
9-(4-fluorophenyl)-1,2,3,4,5,6-hexahydro-1,5-methano-pyrido[1,2a][1,5]diazocin-8-one;
9-(3-fluorophenyl)-1,2,3,4,5,6-hexahydro-1,5-methano-pyrido[1,2a][1,5]diazocin-8-one;
9-(3,5-difluorophenyl)-1,2,3,4,5,6-hexahydro-1,5-methano-pyrido[1,2a][1,5]diazocin-8-one;
9-(2,4-difluorophenyl)-1,2,3,4,5,6-hexahydro-1,5-methano-pyrido[1,2a][1,5]diazocin-8-one;
9-(2,5-difluorophenyl)-1,2,3,4,5,6-hexahydro-1,5-methano-pyrido[1,2a][1,5]diazocin-8-one;
6-methyl-5-oxo-6,13-diazatetracyclo[9.3.1.0^{2,10}.0^{4,8}]pentadeca-2(10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
4,5-difluoro-10-aza-tricyclo[6.3.1.0^{2,7}]dodeca-2(7),3,5-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
6-methyl-5-thia-5-dioxa-6,13-diazatetracyclo[9.3.1.0^{2,10}.0^{4,8}]pentadeca-2(10),3,8-triene;
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
4-chloro-10-azatricyclo[6.3.1.0^{2,7}]dodeca-2(7),3,5-triene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4,5-dichloro-10-azatricyclo[6.3.1.0^{2,7}]dodeca-2(7),3,5-triene;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
5,7,14-triazatetracyclo[10.3.1.0^{2,10}.0^{4,8}]hexadeca-2(10),3,5,8-tetraene;
5,6-dimethyl-5,7,14-triazatetracyclo[10.3.1.0^{2,10}.0^{4,8}]hexadeca-2(10),3,6,8-tetraene;
5-methyl-5,7,14-triazatetracyclo[10.3.1.0^{2,10}.0^{4,8}]hexadeca-2(10),3,6,8-tetraene;
6-(trifluoromethyl)-7-thia-5,14-diazatetracyclo[10.3.1.0^{2,10}.0^{4,8}]hexadeca-2(10),3,5,8-tetraene;
5,8,15-triazatetracyclo[11.3.1.0^{2,11}.0^{4,9}]heptadeca-2(11),3,5,7,9-pentaene;
7-methyl-5,8,15-triazatetracyclo[11.3.1.0^{2,11}.0^{4,9}]heptadeca-2(11),3,5,7,9-pentaene;
6-methyl-5,8,15-triazatetracyclo[11.3.1.0^{2,11}.0^{4,9}]heptadeca-2(11),3,5,7,9-pentaene;
6,7-dimethyl-5,8,15-triazatetracyclo[11.3.1.0^{2,11}.0^{4,9}]heptadeca-2(11),3,5,7,9-pentaene;
7-oxa-5,14-diazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
5-methyl-7-oxa-6,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
7-methyl-5-oxa-6,14-diazatetracyclo[10.3.1.0^{2,10}.0^{4,8}]hexadeca-2(10),3,6,8-tetraene;
4,5-difluoro-11-azatricyclo[7.3.1.0^{2,7}]trideca-2(7),3,5-triene;
4-chloro-5-fluoro-11-azatricyclo[7.3.1.0^{2,7}]trideca-2(7),3,5-triene;
5-chloro-4-fluoro-11-azatricyclo[7.3.1.0^{2,7}]trideca-2(7),3,5-triene;
4-(1-ethynyl)-5-fluoro-11-azatricyclo[7.3.1.0^{2,7}]trideca-2(7),3,5-triene;
5-(1-ethynyl)-4-fluoro-11-azatricyclo[7.3.1.0^{2,7}]trideca-2(7),3,5-triene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
5-fluoro-11-aza-tricyclo[7.3.1.0^{2,7}]trideca-2(7),3,5-triene;
6-hydroxy-5-methoxy-11-aza-tricyclo[7.3.1.0^{2,7}]trideca-2(7),3,5-triene; and
their pharmaceutically acceptable salts and their optical isomers.

11. The pharmaceutical composition according to claim 1 wherein said nicotinic receptor partial agonist is selected from:
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
6-methyl-5-thia-5-dioxa-6,13-diazatetracyclo[9.3.1.0^{2,10}.0^{4,8}]pentadeca-2(10),3,8-triene;
4-fluoro-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
6-methyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7), 3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
6-fluoro-11-aza-tricyclo[7.3.1.0^{2,7}]trideca-2(7),3,5-triene; and
11-aza-tricyclo[7.3.1.0^{2,7}]trideca-2(7),3,5-trien-5-ol, and their pharmaceutically acceptable salts and their optical isomers thereof.

12. A method of treating a mammal which presents with tobacco or nicotine addiction, nicotine withdrawal symptoms, substance abuse or behavioral dependencies, comprising administering to said mammal:
a. a nicotinic receptor partial agonist or a pharmaceutically acceptable salt thereof; and
b. a CB-1 receptor antagonist or a pharmaceutically acceptable salt thereof, wherein the nicotinic receptor partial agonist and the CB-1 receptor antagonist are present in amounts that render the composition effective in the treatment of tobacco or nicotine addiction, nicotine withdrawals symptoms, substance abuse or behavior dependencies.

13. The method according to claim 12, wherein said CB-1 receptor antagonist is selected from 1-[9-(4-chloro-phenyl)-8-(2-chlorophenyl)-9H-purin-6-yl]-3-ethylamino-azetidine-3-carboxylic acid amide; 1-[9-(4-chloro-phenyl)-8-(2-chlorophenyl)-9H-purin-6-yl]-3-ethylamino-azetidine-3-carboxylic acid amide; 1-[9-(4-chlorophenyl)-8-(2-chlorophenyl)-9H-purin-6-yl]-3-isopropylaminoazetidine-3-carboxylic acid amide; 1-{1-[9-(4-chlorophenyl)-8-(2-chlorophenyl)-9H-purin-6-yl]-4-phenylpiperidin-4-yl}-ethanone; {3-[9-(4-chlorophenyl)-8-(2,4-dichlorophenyl)-9H-purin-6-yl]-3-(1a,5a,6a)-azabicyclo[3.1.0]hex-6-yl}-dimethylamine; 6-(1-benzylpyrrolidin-3-yloxy)-9-(4-chlorophenyl)-8-(2,4-dichlorophenyl)-9H-purine; 9-(4-chlorophenyl)-6-(1-cyclohexylazetidin-3-yloxy)-8-(2,4-dichlorophenyl)-9H-purine; 6-tert-butoxy-9-(4-chlorophenyl)-8-(2,4-dichlorophenyl)-9H-purine; 9-(4-chlorophenyl)-8-(2,4-dichlorophenyl)-6-isopropoxy-9H-purine; 1-[9-(4-chlorophenyl)-8-(2,4-dichlorophenyl)-9H-purin-6-yl]-4-propylaminopiperidine-4-carboxylic acid amide; 1-[9-(4-chlorophenyl)-8-(2-fluorophenyl)-9H-purin-6-yl]-4-propylaminopiperidine-4-carboxylic acid amide; 1-[9-(4-chlorophenyl)-8-(2-chlorophenyl)-9H-purin-6-yl]-4-propylaminopiperidine-4-carboxylic acid amide; 1-[9-(4-chlorophenyl)-8-(2-fluorophenyl)-2-methyl-9H-purin-6-yl]-4-isopropylaminopiperidine-4-carboxylic acid amide; 1-[9-(4-chlorophenyl)-8-(2-chlorophenyl)-9H-purin-6-yl]-4-pyrrolidin-1-yl-piperidine-4-carboxylic acid amide; 1-[9-(4-chlorophenyl)-8-(2-chlorophenyl)-9H-purin-6-yl]-4-ethylamino-piperidine-4-carboxylic acid amide; 1-[9-(4-chlorophenyl)-8-(2-chlorophenyl)-9H-purin-6-yl]-4-isopropylaminopiperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 9-[9-(4-chlorophenyl)-8-(2-chlorophenyl)-9H-purin-6-yl]-1-methyl-4-oxa-1,9-diazaspiro[5.5]undecan-2-one; 8-[9-(4-chlorophenyl)-8-(2,4-dichlorophenyl)-9H-purin-6-yl]-1-isopropyl-1,3,8-triazaspiro[4.5]decan-4-one; 1-[9-(4-chlorophenyl)-8-(2-chlorophenyl)-9H-purin-6-yl]-4-(4-fluorophenyl)-piperidin-4-ol; 1-[9-(4-chlorophenyl)-8-(2-chlorophenyl)-9H-purin-6-yl]-4-phenylpiperidin-4-ol; 4-benzyl-1-[9-(4-chlorophenyl)-8-(2-chlorophenyl)-9H-purin-6-yl]-piperidin-4-ol; 4-[9-(4-chlorophenyl)-8-(2-chlorophenyl)-9H-purin-6-yl]-piperazine-2-carboxylic acid methylamide; 9-(4-chlorophenyl)-8-(2,4-dichlorophenyl)-6-(4-pyridin-2-yl-piperazin-1-yl)-9H-purine; and 9-(4-chlorophenyl)-8-(2,4-dichlorophenyl)-6-(4-pyrimidin-2-yl-piperazin-1-yl)-9H-purine; 1-[9-(4-chlorophenyl)-8-(2-fluorophenyl)-9H-purin-6-yl]-4-isopropylamino-piperidine-4-carboxylic acid amide; 1-[9-(4-chlorophenyl)-8-(2-chlorophenyl)-9H-purin-6-yl]-4-isopropylamino-piperidine-4-carboxylic acid amide; 4-amino-1-[9-(4-chlorophenyl)-8-(2-chlorophenyl)-9H-purin-6-y;]-piperidine-4-carboxylic acid amide; 1-[9-(4-chlorophenyl)-8-(2-chlorophenyl)-9H-purin-6-yl]-4-ethylamino-piperidine-4-carboxylic acid amide; 8-[9-(4-chlorophenyl)-8-(2-chlorophenyl)-9H-purin-6-yl]-1-isopropyl-1,3,8-triazaspiro[4.5]decan-4-one; 4-amino-1-[9-(4-chloro-phenyl)-8-(2-chlorophenyl)-9H-purin-6-yl]-piperidine-4-carboxylic acid amide; and 1-[9-(4-chlorophenyl)-8-(2-chlorophenyl)-9H-purin-6-yl]-4-ethylaminopiperidine-4-carboxylic acid amide; and a pharmaceutically acceptable salt thereof of a solvate or hydrate of said compound or said salt.

14. The method according to claim 12, wherein the CB-1 receptor antagonist is selected from 7-(2-chlorophenyl)-8-(4-chlorophenyl)-2-methyl-4-(4-methylpiperazin-1-yl)-pyrazolo[1,5-a][1,3,5]triazine; 7-(2-chlorophenyl)-8-(4-chlorophenyl)-2-methyl-4-(4-pyrimidin-2-ylpiperazin-1-yl)-pyrazolo[1,5-a][1,3,5]triazine; 7-(2-chlorophenyl)-8-(4-chlorophenyl)-4-[(1S,4S)-5-methanesulfonyl-2,5-diazabicyclo[2.2.1]hept-2-yl]-2-methylpyrazolo[1,5-a][1,3,5]triazine; and 7-(2-chlorophenyl)-8-(4-chlorophenyl)-2-methyl-4-[4-(propane-2-sulfonyl)-piperazin-1-yl]-pyrazolo[1,5-a][1,3,5]triazine; 1-[7-(2-chlorophenyl)-8-(4-chlorophenyl)-2-methylpyrazolo[1,5-a][1,3,5]triazin-4-yl]-4-methylaminopiperidine-4-carboxylic acid amide; 1-[7-(2-chlorophenyl)-8-(4-fluorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-4-isopropylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; 1-[7-(2-chlorophenyl)-8-(4-chlorophenyl)-2-methylpyrazolo[1,5-a][1,3,5]triazin-4-yl]-3-methylaminoazetidine-3-carboxylic acid amide; and 1-[7-(2-chlorophenyl)-8-(4-chlorophenyl)-2-methylpyrazolo[1,5-a][1,3,5]triazin-4-yl]-3-dimethylaminoazetidine-3-carboxylic acid amide; 1-{1-[7-(2-chlorophenyl)-8-(4-chlorophenyl)-2-methylpyrazolo[1,5-a][1,3,5]triazin-4-yl]-4-phenylpiperidin-4-yl}-ethanone; 3-[7-(2-chlorophenyl)-8-(4-chlorophenyl)-2-methylpyrazolo[1,5-a][1,3,5]triazin-4-yl]-3-azabicyclo[3.1.0]hex-6-ylamine; 1-[7-(2-chlorophenyl)-8-(4-chlorophenyl)-2-methylpyrazolo[1,5-a][1,3,5]triazin-4-yl]-4-(4-fluorophenyl)-piperidin-4-ol; 4-benzyl-1-[7-(2-chlorophenyl)-8-(4-chlorophenyl)-2-methylpyrazolo[1,5-a][1,3,5]triazin-4-yl]-piperidin-4-ol; 2-[7-(2-chlorophenyl)-8-(4-chlorophenyl)-2-methylpyrazolo[1,5-a][1,3,5]triazin-4-yl]-5-methyl-2,5,7-triazaspiro[3.4]octan-8-one; 2-[7-(2-chlorophenyl)-8-(4-chlorophenyl)-2-methylpyrazolo[1,5-a][1,3,5]triazin-4-yl]-2,5,7-triazaspiro[3.4]octan-8-one; 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; 2-[7-(2-chlorophenyl)-8-(4-chlorophenyl)-2-methylpyrazolo[1,5-a][1,3,5]triazin-4-yl]-6,6-dimethyl-2,5,7-triazaspiro[3.4]octan-8-one; 4-(1-benzylpyrrolidin-3-yloxy)-7-(2-chlorophenyl)-8-(4-chlorophenyl)-2-methylpyrazolo[1,5-a][1,3,5]triazine; 7-(2-chlorophenyl)-8-(4-chlorophenyl)-4-(1-cyclohexylazetidin-3-yloxy)-2-methylpyrazolo[1,5-a][1,3,5]triazine; 7-(2-chlorophenyl)-8-(4-chlorophenyl)-4-isopropoxy-2-methylpyrazolo[1,5-a][1,3,5]triazine; and 4-tert-butoxy-7-(2-chlorophenyl)-8-(4-chlorophenyl)-2-methylpyrazolo[1,5-a][1,3,5]triazine; butyl-[7-(2-chlorophenyl)-8-(4-chlorophenyl)-2-methylpyrazolo[1,5-a][1,3,5]triazin-4-yl]-amine; 7-(2-chlorophenyl)-8-(4-chlorophenyl)-2-methyl-4-piperidin-1-yl-pyrazolo[1,5-a][1,3,5]triazine; [7-(2-chlorophenyl)-8-(4-chlorophenyl)-2-methylpyrazolo[1,5-a][1,3,5]triazin-4-yl]-[2-(4-fluorophenyl)-ethyl]-amine; 7-(2-chlorophenyl)-8-(4-chlorophenyl)-2-methyl-4-morpholin-4-yl-pyrazolo[1,5-a][1,3,5]triazine; and [7-(2-chlorophenyl)-8-(4-chlorophenyl)-2-methylpyrazolo[1,5-a][1,3,5]triazin-4-yl]-(2-morpholin-4-yl-ethyl)-amine; 1-[7-(2-chlorophenyl)-8-(4-chlorophenyl)-2-methylpyrazolo[1,5-a][1,3,5]triazin-4-yl]-4-ethylaminopiperidine-4-carboxylic acid amide; 1-[7-(2-chlorophenyl)-8-(4-chlorophenyl)-2-methylpyrazolo[1,5-a][1,3,5]triazin-4-yl]-3-ethylaminoazetidine-3-carboxylic acid amide; 1-[7-(2-chlorophenyl)-8-(4-chlorophenyl)-2-methylpyrazolo[1,5-a][1,3,5]triazin-4-yl]-3-isopropylaminoazetidine-3-carboxylic acid amide; 3-amino-1-[7-(2-chlorophenyl)-8-(4-chlorophenyl)-2-methylpyrazolo[1,5-a][1,3,5]triazin-4-yl]-azetidine-3-carboxylic acid amide; and 8-[7-(2-chlorophenyl)-8-(4-chlorophenyl)-2-methylpyrazolo[1,5-a][1,3,5]triazin-4-yl]-1-isopropyl-1,3,8-triazaspiro[4.5]decan-4-one; and a pharmaceutically acceptable salt thereof or a solvate or hydrate of said compound or said salt.

15. The method according to claim 12, wherein said CB-1 receptor antagonist is selected from 3-(4-chlorophenyl)-2-(2-chlorophenyl)-7-(4-methyl-piperazin-1-yl)-pyrazolo[1,5-a]pyrimidine; 3-(4-chlorophenyl)-2-(2-chlorophenyl)-7-(4-pyrimidin-2-yl-piperazin-1-yl)-pyrazolo[1,5-a]pyrimidine; 3-(4-chloro-phenyl)-2-(2-chlorophenyl)-7-[(1S,4S)-5-methanesulfonyl-2,5-diazabicyclo[2.2.1]hept-2-yl]-pyrazolo[1,5-a]pyrimidine; and 3-(4-chlorophenyl)-2-(2-chlorophenyl)-7-[4-(propane-2-sulfonyl)-piperazin-1-yl]-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; 1-[3-(4-chlorophenyl)-2-(2-chlorophenyl)-pyrazolo[1,5-a]pyrimidin-7-yl]-3-ethylaminoazetidine-3-carboxylic acid amide; 3-amino-1-[3-(4-chlorophenyl)-2-(2-chlorophenyl)-pyrazolo[1,5-a]pyrimidin-7-yl]-azetidine-3-carboxylic acid amide; 1-[3-(4-chlorophenyl)-2-(2-chlorophenyl)-6-methylpyrazolo[1,5-a]pyrimidin-7-yl]-3-ethylaminoazetidine-3-carboxylic acid amide; 1-[3-(4-chlorophenyl)-2-(2-chlorophenyl)-pyrazolo[1,5-a]pyrimidin-7-yl]-3-isopropylaminoazetidine-3-carboxylic acid amide; 1-[3-(4-chlorophenyl)-2-(2-chlorophenyl)-5,6-dimethylpyrazolo[1,5-a]pyrimidin-7-yl]-3-ethylamino-azetidine-3-carboxylic acid amide; 1-[3-(4-chlorophenyl)-2-(2-chlorophenyl)-pyrazolo[1,5-a]pyrimidin-7-yl]-3-methylaminoazetidine-3-carboxylic acid amide; 1-[3-(4-chlorophenyl)-2-(2-chlorophenyl)-5-methylpyrazolo[1,5-a]pyrimidin-7-yl]-3-ethylaminoazetidine-3-carboxylic acid amide; 1-{1-[3-(4-chlorophenyl)-2-(2-chlorophenyl)-pyrazolo[1,5-a]pyrimidin-7-yl]-4-phenylpiperidin-4-yl}-ethanone; 3-[3-(4-chlorophenyl)-2-(2-chlorophenyl)-pyrazolo[1,5-a]pyrimidin-7-yl]-3-(1a,5a,6a)-azabicyclo[3.1.0]hex-6-ylamine; 1-[3-(4-chlorophenyl)-2-(2-chlorophenyl)-pyrazolo[1,5-a]pyrimidin-7-yl]-4-(4-fluorophenyl)-piperidin-4-ol; 4-benzyl-1-[3-(4-chlorophenyl)-2-(2-chlorophenyl)-pyrazolo[1,5-a]pyrimidin-7-yl]-piperidin-4-ol; 8-[3-(4-chlorophenyl)-2-(2-chlorophenyl)-pyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-2,5,7-triazaspiro[3.4]octan-8-one; 8-[3-(4-chlorophenyl)-2-(2-chlorophenyl)-6-methylpyrazolo[1,5-a]pyrimidin-7-yl]-1-isopropyl-1,3,8-triazaspiro[4.5]decan-4-one; 2-[3-(4-chlorophenyl)-2-(2-chlorophenyl)-pyrazolo[1,5-a]pyrimidin-7-yl]-5-methyl-2,5,7-triazaspiro[3.4]octan-8-one; 7-(1-benzylpyrrolidin-3-yloxy)-3-(4-chlorophenyl)-2-(2-chlorophenyl)-pyrazolo[1,5-a]pyrimidine; and 3-(4-chlorophenyl)-2-(2-chlorophenyl)-7-(1-cyclohexylazetidin-3-yloxy)-pyrazolo[1,5-a]pyrimidine; 1-[3-(4-chlorophenyl)-2-(2-chlorophenyl)-pyrazolo[1,5-a]pyrimidin-7-yl]-4-ethylaminopiperidine-4-carboxylic acid amide; 1-[3-(4-chlorophenyl)-2-(2-chlorophenyl)-pyrazolo[1,5-a]pyrimidin-7-yl]-4-isopropylaminopiperidine-4-carboxylic acid amide; and 1-[3-(4-chlorophenyl)-2-(2-chlorophenyl)-pyrazolo[1,5-a]pyrimidin-7-yl]-3-ethylaminoazetidine-3-carboxylic acid amide; and a pharmaceutically acceptable salt thereof or a solvate or hydrate of said compound or said salt.

16. The method according to claim 12, wherein said CB-1 receptor antagonist is selected from 5-(4-chloro-phenyl)-3-(5-cyclohexyl-1H-imidazol-2-yl)-1-(2,4-dichloro-phenyl)-4-methyl-1H-pyrazole; 5-(4-chloro-phenyl)-3-(2-cyclohexyl-3H-imidazol-4-yl)-1-(2,4-dichloro-phenyl)-4-methyl-1H-pyrazole; 5-(4-chloro-phenyl)-1-(2,4-dichloro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-4-methyl-3-[1-(1-phenyl-ethyl)-1H-imidazol-4-yl]-1H-pyrazole; 5-(4-chloro-phenyl)-1-(2-fluoro-phenyl)-4-methyl-3-[1-(1-methyl-1-phenyl-ethyl)-1H-imidazol-4-yl]-1H-pyrazole; 5-(4-chloro-phenyl)-1-(2-chloro-phenyl)-3-[1-(2,2-dimethyl-tetrahydro-pyran-4-yl)-1H-imidazol-4-yl]-4-methyl-1H-pyrazole: 5-{2-(2,4-dichloro-phenyl)-4-methyl-5-[1-(1-methyl-1-phenyl-ethyl)-1H-imidazol-4-yl]-2H-pyrazol-3-yl}-2-methoxy-pyridine; and 1-(2-chloro-phenyl)-5-(4-chloro-phenyl)-4-methyl-3-[1-(1-methyl-1-phenyl-ethyl)-1H-imidazol-4-yl]-1H-pyrazole; and a pharmaceutically acceptable salt thereof or a solvate or hydrate of the compound or the salt.

17. The method according to claim 12, wherein said CB-1 receptor antagonist is selected from 1-[5-(4-chloro-phenyl)-1-(2-chloro-phenyl)-4-methyl-1H-pyrazol-3-yl]-2-piperidin-1-yl-ethanone; 1-[5-(4-chloro-phenyl)-1-(2-chloro-phenyl)-4-methyl-1H-pyrazol-3-yl]-2-morpholin-4-yl-ethanone; 1-[5-(4-chloro-phenyl)-1-(2-chloro-phenyl)-4-methyl-1H-pyrazol-3-yl]-2-[4-(1-methyl-1H-pyrrole-2-carbonyl)-piperazin-1-yl]-ethanone; 1-[5-(4-chloro-phenyl)-1-(2-chloro-phenyl)-4-methyl-1H-pyrazol-3-yl]-2-[4-(1-methyl-cyclopropanecarbonyl)-piperazin-1-yl]-ethanone; N-(1-{2-[5-(4-chloro-phenyl)-1-(2-chloro-phenyl)-4-methyl-1H-pyrazol-3-yl]-2-oxo-ethyl}-piperidin-4-yl)-2,2,2-trifluoro-acetamide; 1-[5-(4-chloro-phenyl)-1-(2-fluoro-phenyl)-4-methyl-1H-pyrazol-3-yl]-2-morpholin-4-yl-ethanone; 1-[5-(4-chloro-phenyl)-1-(2-fluoro-phenyl)-4-methyl-1H-pyrazol-3-yl]-2-piperidin-1-yl-ethanone; 1-[5-(4-chloro-phenyl)-1-(2-fluoro-phenyl)-4-methyl-1H-pyrazol-3-yl]-2-(4-trifluoroacetyl-piperazin-1-yl)-ethanone; 1-[1-(2-chloro-phenyl)-5-(4-chloro-phenyl)-4-methyl-1H-pyrazol-3-yl]-2-pyrrolidin-1-yl-ethanone; 1-[1-(2-chloro-phenyl)-5-(4-chloro-phenyl)-4-methyl-1H-pyrazol-3-yl]-2-[1,4]oxazepan-4-yl-ethanone; and 1-[5-(4-chloro-phenyl)-1-(2-chloro-phenyl)-4-methyl-1H-pyrazol-3-yl]-2-(1-oxa-8-aza-spiro[4.5]dec-8-yl)-ethanone; and a pharmaceutically acceptable salt thereof, or a solvate or hydrate of the compound or the salt.

18. The method according claim 12, wherein said CB-1 receptor antagonist is selected from 2-(benzyl-isopropyl-amino)-1-[1-(2-chloro-phenyl)-5-(4-chloro-phenyl)-4-methyl-1H-pyrazol-3-yl]-ethanol; 1-[5-(4-chloro-phenyl)-1-(2-chloro-phenyl)-4-methyl-1H-pyrazol-3-yl]-2-(3,5-dimethyl-piperidin-1-yl)-ethanol; 1-{2-[1-(2-chloro-phenyl)-5-(4-chloro-phenyl)-4-methyl-1H-pyrazol-3-yl]-2-hydroxy-ethyl-}4-isopropylamino-piperidine-4-carboxylic acid amide; 1-[5-(4-chloro-phenyl)-1-(2-chloro-phenyl)-4-methyl-1H-pyrazol-3-yl]-2-(3,3-dimethyl-piperidin-1-yl)-ethanol; 1-[5-(4-chloro-phenyl)-1-(2-chloro-phenyl)-4-methyl-1H-pyrazol-3-yl]-2-piperidin-1-yl-ethanol; and 1-[5-(4-chloro-phenyl)-1-(2-chloro-phenyl)-4-methyl-1H-pyrazol-3-yl]-2-morpholin-4-yl-ethanol; and a pharmaceutically acceptable salt thereof, or a solvate or hydrate of the compound or the salt.

19. The method according to claim 12 wherein said CB-1 receptor antagonist is selected from 2-[5-(4-chloro-phenyl)-1-(2-chloro-phenyl)-4-methyl-1H-pyrazol-3-yl]-4-cyclohexyl-morpholine; 2-[5-(4-chloro-phenyl)-1-(2-chloro-phenyl)-4-methyl-1H-pyrazol-3-yl]-4-(propane-2-sulfonyl)-morpholine; 2-[5-(4-chloro-phenyl)-1-(2-chloro-phenyl)-4-methyl-1H-pyrazol-3-yl]-4-(toluene-4-sulfonyl)-morpholine; 1-{2-[1-(2-chloro-phenyl)-5-(4-chloro-phenyl)-4-methyl-1H-pyrazol-3-yl]-morpholin-4-yl}-2-methyl-propan-1-one; and 2-[1-(2-chloro-phenyl)-5-(4-chloro-phenyl)-4-methyl-1H-pyrazol-3-yl]-4-(4-trifluoromethyl-benzyl)-morpholine; and a pharmaceutically acceptable salt thereof or a solvate or hydrate of the compound or the salt.

20. The method according claim 12, wherein said CB-1 receptor antagonist is selected from 1-[1-(4-chloro-phenyl)-2-(2,4-dichloro-phenyl)-5-methyl-1H-imidazol-4-yl]-2-piperidin-1-yl-ethanone and 1-[1-(4-chloro-phenyl)-2-(2,4-dichloro-phenyl)-5-methyl-1H-imidazol-4-yl]-2-morpholin-4-yl-ethanone; and a pharmaceutically acceptable salt thereof, a or a solvate or hydrate of the compound, or the salt.

21. The method according to claim 12, wherein the nicotine partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
9-vinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2-a][1,5]diazocin-8-one;
3-benzyl-9-bromo-1,2,3,4,5,6-hexahydro-1,5-methano-pyrido[1,2-a][1,5]diazocin-8-one;
3-benzyl-9-chloro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-ethynyl-1,2,3,4,5,6-hexahydro-1,5-methano-pyrido[1,2a][1,5]diazocin-8-one;
9-(2-propenyl)-1,2,3,4,5,6-hexahydro-1,5-methano-pyrido[1,2a][1,5]diazocin-8-one;
9-(2-propyl)-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
9-(4-fluorophenyl)-1,2,3,4,5,6-hexahydro-1,5-methano-pyrido[1,2a][1,5]diazocin-8-one;
9-(3-fluorophenyl)-1,2,3,4,5,6-hexahydro-1,5-methano-pyrido[1,2a][1,5]diazocin-8-one;
9-(3,5-difluorophenyl)-1,2,3,4,5,6-hexahydro-1,5-methano-pyrido[1,2a][1,5]diazocin-8-one;
9-(2,4-difluorophenyl)-1,2,3,4,5,6-hexahydro-1,5-methano-pyrido[1,2a][1,5]diazocin-8-one;
9-(2,5-difluorophenyl)-1,2,3,4,5,6-hexahydro-1,5-methano-pyrido[1,2a][1,5]diazocin-8-one;
6-methyl-5-oxo-6,13-diazatetracyclo[9.3.1.0^{2,10}.0^{4,8}]pentadeca-2(10),3,8-triene;
5-oxo-6,13-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
4,5-difluoro-10-aza-tricyclo[6.3.1.0^{2,7}]dodeca-2(7),3,5-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene-4-carbonitrile;
6-methyl-5-thia-5-dioxa-6,13-diazatetracyclo[9.3.1.0^{2,10}.0^{4,8}]pentadeca-2(O),3,8-triene;
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,10}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
4-chloro-10-azatricyclo[6.3.1.0^{2,7}]dodeca-2(7),3,5-triene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4,5-dichloro-10-azatricyclo[6.3.1.0^{2,7}]dodeca-2(7),3,5-triene;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
5,7,14-triazatetracyclo[10.3.1.0^{2,10}.0^{4,8}]hexadeca-2(10),3,5,8-tetraene;
5,6-dimethyl-5,7,14-triazatetracyclo[10.3.1.0^{2,10}.0^{4,8}]hexadeca-2(10),3,6,8-tetraene;
5-methyl-5,7,14-triazatetracyclo[10.3.1.0^{2,10}.0^{4,8}]hexadeca-2(10),3,6,8-tetraene;
6-(trifluoromethyl)-7-thia-5,14-diazatetracyclo[10.3.1.0^{2,10}.0^{4,8}]hexadeca-2(10),3,5,8-tetraene;
5,8,15-triazatetracyclo[11.3.1.0^{2,10}.0^{4,9}]heptadeca-2(11),3,5,7,9-pentaene;
7-methyl-5,8,15-triazatetracyclo[11.3.1.0^{2,11}.0^{4,9}]heptadeca-2(11),3,5,7,9-pentaene;
6-methyl-5,8,15-triazatetracyclo[11.3.1.0^{2,11}.0^{4,9}]heptadeca-2(11),3,5,7,9-pentaene;
6,7-dimethyl-5,8,15-triazatetracyclo[11.3.1.0^{2,11}.0^{4,9}]heptadeca-2(11),3,5,7,9-pentaene;
7-oxa-5,14-diazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
5-methyl-7-oxa-6,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
7-methyl-5-oxa-6,14-diazatetracyclo[10.3.1.0^{2,10}.0^{4,8}]hexadeca-2(10),3,6,8-tetraene;
4,5-difluoro-11-azatricyclo[7.3.1.0^{2,7}]trideca-2(7),3,5-triene;
4-chloro-5-fluoro-11-azatricyclo[7.3.1.0^{2,7}]trideca-2(7),3,5-triene;
5-chloro-4-fluoro-11-azatricyclo[7.3.1.0^{2,7}]trideca-2(7),3,5-triene;
4-(1-ethynyl)-5-fluoro-11-azatricyclo[7.3.1.0^{2,7}]trideca-2(7),3,5-triene;
5-(1-ethynyl)-4-fluoro-11-azatricyclo[7.3.1.0^{2,7}]trideca-2(7),3,5-triene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
5-fluoro-11-aza-tricyclo[7.3.1.0^{2,7}]trideca-2(7),3,5-triene;
6-hydroxy-5-methoxy-11-aza-tricyclo[7.3.1.0^{2,7}]trideca-2(7),3,5-triene
and a pharmaceutically acceptable salt and an optical isomer thereof.

22. The method according to claim 12, wherein the nicotine partial agonist is selected from
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-fluoro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
6-methyl-5-thia-5-dioxa-6,13-diazatetracyclo[9.3.1.0^{2,10}.0^{4,8}]pentadeca-2(10),3,8-triene;
4-fluoro-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
6-methyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol; and the pharmaceutically acceptable salts and optical isomers thereof.

23. The method according to claim 12, wherein the nicotinic receptor partial agonist and the CB-1 receptor antagonist are administered substantially simultaneously.
